SEQUENCE LISTING (8 sequences)

SEQ ID NO: 1 - DNA, 21 bp, Artificial Sequence
  Description of Artificial Sequence FCGR3A specific primer.
atatttacagaatggcacagg

SEQ ID NO: 2 - DNA, 20 bp, Artificial Sequence
  Description of Artificial Sequence FCGR3A specific primer.
gacttggtacccaggttgaa

SEQ ID NO: 3 - DNA, 32 bp, Artificial Sequence
  Description of Artificial Sequence Amplification primer.
atcagattcgatcctacttctgcagggggcat

SEQ ID NO: 4 - DNA, 32 bp, Artificial Sequence
  Description of Artificial Sequence Amplification primer.
acgtgctgagcttgagtgatggtgatgttcac

SEQ ID NO: 5 - DNA, 22 bp, Artificial Sequence
  Description of Artificial Sequence Amplification sense primer.
ggaaaatcccagaaattctcgc

SEQ ID NO: 6 - DNA, 27 bp, Artificial Sequence
  Description of Artificial Sequence Amplification antisens primer.
caacagcctgactacctattacgcggg

SEQ ID NO: 7 - protein, 254 aa, Artificial Sequence
  Description of Artificial Sequence Amino acid sequence of human FCGR3A158F.
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 8 - DNA, 22685 bp, Artificial Sequence
  Description of Artificial Sequence Nucleic acid sequence of human FCGR3A158F.
cagcctggctgacacagtgagacctcatctctaaaaaaaaaagcaagcagaatattttcttaaaaggcaattatattccttcttggccaggcccagtggctcacacctgtaatcccagcactttgggaggccgagatgggtggatcacctgaggtcaggagttcgagaccagcctggccaacatggcgaaaacccgtgtctactaaaaatacaaaaattagctgggcatgggggcatatgcctgtaatcccagctacttgggaggctgagacaggagaatcgcttgtacccgggaggcagagattgcagtgagccgagatcatgccactgcactccagccttggcgacagagtgaggctttgtctaaaaaaaaaaaggtattttttgcctctctgttggtaccaattgttaaattctttgtggaccactgatgcttaccaaaaaaaaaaaaaaaaaaagtgggggcatcatatttcctctagttgacattaagacacagtaatttagccagaggagatcttagcaaacatacagtccacactccactttctcatttcatgattgtagagactgagatctagacaatttaatcggtggtcaccctgggtgacatagctaggtctagagctcctggtctccaggtcagcatttctttcttcttcattaaatgtcaagtttcctcccctgttcattattagctccttccagaaagagagtttcttatctttttagtaggtactcagtaaataccaaggtattcactaggatgcctttggatgaaggtaacaagccctgacttaaattggcttaaacagcagggaaatttacttgaaattgtaagaaatctgggttggttgcgttagaagctcagtgatgtcaccaagaccatattctatccctccactctgtcctccttggctatttggcattgacctcagactggctgccttcaaaatcttaggttttgccagcagaacctaggacaaaatgagcccttgttcatgtacagtgggagagagagatcatctctcccaaacatggcactcccctctaccagattggccctatttaggacaaagttccgtcttctcccacttaaccaataaaagccaggggaatgctacaccctgagtggcttagatcagtcaagatccacctctgcatatgagggtgattcctgaatagaatcaaggttatattagaagggagggagagggatggatatcaggctagtacatcatattctatttgttgagttaactgagtcatagcaattgttgagttggaaaaaactcagaacctactgtggattcaagttcaagaaatcattctttcctacatacaacagcattgctctgtagccctgagctaagagagcatcacgaaatacagtcttcttgctgtttataatcgtaagcaaactcttggacctgggaggggatgaatggataatgtctgtctgacttgcttctttctagttagtaccaactacctcccttcttcctgtgattgttcttagaataggataaaaaatcttcccttccctagatcttacagtctccccttcccccaggcctttctatttttcaggattttactctaatcacaccaccgaagaatcaagaaatctttaaagtgtattagagtagctagttgtggcagcactaaaacacggctgcaaattctttgacactctctccatcaagaaatgaggcctacatcctctacccttgaatctgggtgggcttataacttctggtgattagactacagcagaaagagaagctgtatagcttcctaaatgttataaagtcttaaggatagctgccagcaagaccttaggacaaaatgagcccttgttcacgtccagtgggagagagagagagagaccctctctcccaaatatggcaatcctctctacctataacatttacataaatgttataggttaaacgttccacaacaaactaagtactatttaacatcaagaggaaaaagagacaggagaaagggttaataagcctgttgatgaggatctaagaagaacaaaggaggcctggtttgggcctggctacccgttggtcttgcaaagaagagtctgaggtggcagagccttcagtggcagatgccaaattatcatcatgagtgactgcaagacagtgtcagctaagatagccatttcaagctgctgaaggccttctcttttagtcgtggagtcctgtgataagaactgaaagttggaagagtgtgcttgtctgtggccttatttggtcggatgcagtctttatcatttttaatttgtttcttagaacattttatcttgttggccaaatgccctacgaaatataaaatggagtctttttctaagatggagttagttatgtcaagggtcctttatacagtcttcatcctttttcctggcatacaactcctaaaatccttagaatctccaaagtgatgtcttttggtgtgctaatgaggtaactgatggctggcagctcttaggtagcttcataacaggggctgggcacaagaaagatcatggcaaggtcagaggattggggctttcagctccaccctccaaactccctctgggaagtggagaggggctgaaggttgaattgatcaccaatagccaatgacttaattaatcattcctaagtaataaagctcccataaaaacccaaaaggacagggtttggagatcctccagagagccgaacacagagaggttcttggagggtagtgcaccagagggcatggaagctccaagccccttcccacaggtcttgccctatgtactctttacttgtgtcctttgtaatattctttatcacaaactgataaatgtaaatgtttccctgagtactgagagccactctagcaaattaattgaacccaagatgcaggtggtgggaacccccatttataactggttggtcaaaagcacaggtaaaacaacctggggcttcatcctggagtatcagaagtgtcttgtgagactgagcccttcacttgtgtcacctgatgctatttccagttagatagtgttggaattcaattgaatttgagcagaagtcccaatccccagacctgtagttgtcagtgacctcttaggaactgggctgcacagcaggaagtgaggggcaggtggggagcaaagctttatctgtatttacagtagatccccatggctcacatcaccgcctgagctcctcctcctatcagatcagctgtggcattaaattatcacaggagcatgaaccctattgtgaagtacgcatgcaagggatctaggttgcattctccttatgagaatctaatgcctgatgacctggcattgtctcccatcaccctagatgggactgtctagttgcaagaaaacaagctcggggctccccctgattctacattatggtaagttgtataattacttcattaaatattacactgtaataataatagaaatgaagtgcacaataaatgtaatgcacttgaatcatcctgaaaccatcccccacccccctacccctgtccgaggaaaaattgtcttccatgaagccaatccctggtgccaaaaattttggagaccactggattagaagacacccagttggtgcccactgctgaattgcttgcttgcttgcttgcttgcttgccagtggagagaaatccccacatatctgttgtcagaaatgtgttgtgagagcatagtgggaggaactgagtttgttttttctacagttacagcaataggtaactggaattcaactgctggactataccaaagactgccaggccagcctacctttctcacagccttcttgactacctgtcttggatgagctcactgaaagcccacataccttcattctagcatttcctcagtctggttgagctgctttggaggtaatacaggttgtaggacttccctccactcctgctcaggaccgttttcagcaggctaatcagacagcagttggcactgagtacaactggagaaatgttatcagcactgaagactgctccagctacaaatgcacagcgacccacctcagctggacctttggcattgcttggcaatccttacaactgtttggaattcttggtaattcttgtacatacaaacagtcggctccttctcccatgcaccacaatggccattctcaactctggtgttctcaaacccccaccacaactgttacccactctttctcgacaagtgtctttgactcctccttaaccaagaaaatcaggaccagcagatgtggatggtcactcaacatctgaaaatggatttgcatatgtaccccctcagctcctgccttcagctcagagcgagaggtaatccgtatccagttcacagccaactccctgtccatgtcccattcccatctcctcaggacccacacttgcttctctaggtgttccgtccctgttaggcatccaacttctcccaccctgctggcttcttcccaaagacctataaccaagctcgtatctttcacttaaaagaaaacaaaaactcccattctcttgtaactacctttgcagaattgtgtctgaaacagtgagagagatctaacttaattgactccatcttgcttctaacctccaagctgtctttcctcattcttgggcataggctgaactaactttgggagaaacttagtttatagtttgtggtttaaagcaaagatgataacagccctttcccagggcagacctcctttttttctgaagactagattgtctttgtaggactaacattagccacaagattggaaattatggtttaggaatcatgcaggtggaggctacaagattctgacccttcctaagcactgatcctaagatcggtgcttgagatattttgcagaccctgcacttgatggatcacctggcaacacccagatcaataaactggctcatctgatcttgtggtgcccacccaggaactgactcagaacaagaagacagcttcaacttcctgtgatttcatccctgaccaatcaacactcctggctcactggcttcccctccaccaaccaagttgtccttaaaaactctgctccccgaatgctctggaagactgatttgagtaataataaaactccagtctctggctcagtcagctctgcatgaattactctttctctattgcaattcccctgtcttgatgaatcagctctgtctagttaccatcctccacttctcctttcttattgtgtcacttagggctctgggttataaacaactttatcagaatccagatcttttaagtagaggaaaaagatttattggatggaaactagaggaggtgagcccatcccactgctgtgatgactgggacccaaccatctccctctcctagaagtgaatctcccttatgagtaaacaagtgtcacttattcaggattcatctcagaagagactctaatgggccaaacctcagttatatgcctgtcctctgtctgcctgtatcagttagctagcacttttataacaaagtaccacagactgggtggcttaaacaacaaaaatgtattttcttacagttcttgaggctgcaagtccaagatcaaggtgttgacagggttggtttcttttaattttttttttaaaaattttattttagatttaggggtatgcgtgcaggtttgctacataggtaaactcctatcacgggggtttgttgcacacattatttcatcacccatgtactaagcctagttactcaatagttattttttccgatcctctccctcttcctaccctccaccctcaagtaggccccagtgtgtctgttgttcttcctttgagtccatagggttggtttcttctgaggcctctctccttggcttgtaggtgtccatcttctccctgtatgggtctgtgtcccaactaacaaggacatcagtcatattggactagagcccacctaatgattttattttaacttaattacctctttaaaggctctatatccacatatagtcacatcctgaggtatagggggttccacatgtggacttcaacatatgaactgggagagacaaaatttagtccttaatagtgccccaaagtggggaaaaggaagatctggaccctcgggtttccatagtagaaagcaatcactgctttctattaagtactcacagtggggcttctccagaaagaatgatatgctaataagaaggggaggaggaagtgatcctggacagccagatgatatgtgcactattccttcataatggagattctgaagaggagaagcacttgactaaacactttttcattcctactccgtcttcaaccaaaagctgtcaaacttctgtttctcagccccagcccctgaaattgctcaggaaaaggtcattaatagttccttgattgccatatttcaatcaaactcttgtttgaattcttttctacaacattaatactgttactgttgactactccttccttgaagatctgttcccactaaacttccttgttccctcctcttcagcccctcctatacaaactcctttgtcagctatttttcctgtgcacgcttcaaaaatgtttgcatgtcaagtttctgtcattgactctctcctcttctcctctccctctcaatccctccttcctttccctcactgtttccctttaattctctctcaatactcttacagtttcagagatcttatccttactttatcttaacctaggatctctggatggattcaaatagagcttcttaaattaaaggaaacataatgtgtatatttgcatcctttcttgggagaaggcccaaaggtttttatcagaggtttgaaacctcaaccgtgttggtgcctcctaaattgtgtcttttgtcaagacctgtcttctgagttccagggccatgtgtctcactgcctactggaaatcttcacctgaaaccttcacagctacctcaaactcaataacatcaaaagctgaaatcattgtctctccctcccaaagcctgctcatcttcccatttttcttttgtccatgaaagctactgccatcctccttatcacccaaattagaaatccgagcatcacccagacctctcccccttcatcacccctcagccaatcactcaccaagtcttgtccatccttccttcctaacttctctcctggatgcttccattgcatatccactttttaaacagagtggctcttgtctcaactagactgttgaaataatcttccaacttttccctccaccttccatctctctcccctctaactcattccttggactgctgtcagagtacttttcataaaatataaaacagatcttgtgattccccagtctaaagcctttttattagttcccattaccttttagaataaaatatgtactgttcatcctgacacacaaaactcttcgtgataaatactaattgagtgcctagtatgtgcctgcccttgtgctaaatgttgagggtacaggggtaaacaaggtgaacagcttccctgctctccaagacctttcagtccacaaatgcaatgagtttacagaggagaagcacaagctcctaaaggagttggggtggggttgggggtcagaacctaatttagaaaattgaggaaggtctcaacctcccatcttgcatttacaatagtaatcagcaggtgtggtaccaaatatggaaccaacaattttatctgcattatctcatttaagccatgagtgccattattgttagcctcactttacagataaggaaactgagggctagaaggttaaataagtggcagagttgggatttcctccagattcctgtgagacccagacatcttaatccttttggaacctgtgcttctcctttgtagtactcactacacttgtggaactacatccaactacacttgtggaactacagccagctctgcaaacatgacagtctacttcactccaagtctttgctcatgctgctcctcttgcctggaatgcctatttctctcaaaaatcttcctgctgaatattttgcgatctaattaaagtgttctctcttccatgtacactcctccctcagatagaattagccactgtcttctttgtgcatacacagcatttcataaatactgtcacagtccctctagcacttcaaatacttatctgatgttctccccctaagaaactgtaagtcctagaggatgacaatcaactgaattccatagtcagaaacttctgctgtgcctggccttccaatgagaaaaggagagaagaggaggggaaggaagaaaaagggaaggagaagaaagaaaagcaaacatgaagataaacacttcaatatatgatatcccaagaccatctacccttttgtaaaaattttgctttttttttttcccccccaagagtcagggtctcactctgtcgcccaggctagagtgcagtgccatgaacataactcactgtagcctctaactccggggctcaagcaatcctcctgcctcagcctcctgggtagctgggactacaggcatgcaccaccacatctggctattattattattactatattagtagagatggggtctttctatgttgcctaggctggtctcaaattcctggcctcaagcaattcttccacctcacattggccttccaaagtgctgggattacaataagccaccataggccaaaattttgcattttatccattactgtaaaattaacccttagaaatccaacaacactcaatttgagaattgttcaacaaccacttaatgaaaaccccctgaaagcttcccatcctgttgcagtccctttctctcctcctgtgctctctcctcttcttcctatctagcccacccttttggcagctaagaattcctccctccattggagagccacagaccaaagaggagtcaaataagaaaataagacctcaaagaaggaaaacaaagtgaaggccttgcatcagaagtcacgtggcagaaagccacctggatatctgaaaagaagaaagaattgagggatatccgctttttgcctcagagaccatccttagccctgaaggctttgtttctgctttaggtttcccagataagcatccgaagtgctacagcaaggaactttaagtttccagatacttgtctggattttgcaaggcgtagatgagtcacttgagaaggagaactggaatggctgcctgggttcatttccattgtccaatccaagggcctgtggagaaggggctgctgcaagactctgtgtgtggcggggggaggggtgggtacgtggatggcaatgggaggatcaattaactccacccaggagccaaatgaaacacacaaataaaaaacaaaacctgagtagtggtttttaggtcattctggagtagaaagagcattcatttatagcaaaggttggcgggcacctgtgtcagcccctgcctccactccacccctaacaagtatcaggtgcccacacgggcctgctgctcgcctcctgggcttttctaagccaggtgagacctgtcccagatgtccacgaatccactgggggagtggcactatcaagcagagtcatctgattttctgcctgggacctggaccattgtgagagtaaccaacgtggggttacgggggagaatctggagagaagagaagaggttaacaaccctcccacttcctggccacccccctccaccttttctggtaaggagccctggagccccggctcctaggctgacagaccagcccagatccagtggcccggaggggcctgagctaaatccgcaggacctgggtaacacgaggaaggtaaagagttcctgtcctcgcccctccccacccccaccttttctgtgatcttttcagcctttcgctggtgacttgttcttccagggcccatttctctaccctacctgggtttcttctaacctggaaatctaatgatcaaatcacactaaaaagtcagtagctcctgtggattacatatcccaggagcatatagattttgaattttgaattttgaaagaaattctgcgtggagataatattgaggcagagacactgctagtggtctgaagatttgaaaggaccactttctgtgtgcaggcagggcctcagctggagatagatgggtctgggcgaggcaggagagtgacaagttctgaggtgaaatgaaggaagccctcagagaatgctcctcccaccttgaatctcatccccagggtctcactgtcccattcttggtgctgggtggatccaaatccaggagatggggcaagcatcctgggatggctgagggcacactctggcagattctgtgtgtgtcctcagatgctcagccacagacctttgagggagtaaagggggcagacccacccaccttgcctccaggctctttccttcctggtcctgttctatggtggggctcccttgccagacttcagactgagaagtcagatgaagtttcaagaaaaggaaattggtgggtgacagagatgggtggaggggctggggaaaggctgtttacttcctcctgtctagtcggtttggtccctttagggctccggatatctttggtgacttgtccactccagtgtggcatcaggggctggggaaaggctgtttacttcctcctgtctagtcggtttggtccctttagggctccggatatctttggtgacttgtccactccagtgtggcatcatgtggcagctgctcctcccaactgctctgctacttctaggtaagtcagggtctccctggttgagggagaagtttgagatgccttgggttcagcagagaccccttttcaggctacgaatgagactcccacgaagggatgggacccctcaccacatctatagctgtggattgagctcctaggacaagccaagatggggctagaaatgaggagaatgctggttccaattggggcatactcatgagtgaggccagtcacttcacccctctgggtcccagaatcactctgtggaaccaaagagcttcgactagatggtccctagggtctgtctctttcagtttgacattccagggttctcctctatgattttcaatttctaccctttcttgtggggatatgggttgaggctctttctgtagcttggttcagggaaattcaacctgtacccttaatttgtgagtttgcacagggagcaaggggtaagggagcagtgttgaaaatagggatttgtgttgacagtggcgcaagaggcatgaacagtggagaccagagagcaggtagcaaggtttccaccagaaacatcctgattcttgggaaaattgggctcctggggcagaggagggcaggggagttttaaactcactctatgttctaatcactctgatctctgcccctactcaatatttgatttatcttttttcttgcagtttcagctggcatgcggactggtgagtcagcttcatggtcttggattgacccagtggggcacatatggggacaaaggccataagatattgggaaatgcttgttgaatgggaaaatgctgatgtggggttagcagggatagttcctccaacacagcagaacttggccctgtgcttctctggccagctttccttaagatactgaacaggccaaaaatggggccaagatgctctaagactgagccaccaagcatgggtttgcaatgagctcattctggctttgaggctccctgggaatggcagtgtagagcctgctcctctccctgtcctcaccccacattatcttggctcctcagaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagtcgacgacagtggagagtacaggtgccagacaaacctctccaccctcagtgacccggtgcagctagaagtccatatcggtgagttgatgaaggggaagaggaaaatcaccaataaagggtgaaacaaagggtcctgaaatacttggtaagagccagagatgatattcttagagataaaagctaagatgagatgatgtgtggtcccactgaatggtatcagagttgtagtcctagctctaagtaggtcttgggcaaaatgtcaaagcctgtcagacagtagatataggactgctgcattgcacaattccaagaatccccatatggagtgcatacaatgtgaatgtgtcatgtgaaggttaggccatggcatagatgctcaataatagttatttatatatttattttcattttttttaattttattttttgagacagagtatcactctgtcacccaggctggagtgcaatgcggcaatctcagctcactgcaacttctgcccccttgggttgtagtgattctcctgcctcagcctcccgagtagctgagattacaggcacccgccaccacgcccagctaatttttgtatttttagtagagacagggtttcaccatgttggtcagtctggtctcaaactcctgacctcaggtgattcaccagccttggcttcccaaagtgctgggactacaggcgtgagccaccacacctggccaataatatttattgaataaattaatgaatttggtgttaggacctcaatctccttctcgctctcagacatgtaatgccctaagccacctcccaaagcaatcctagtggcctagcatcatatctttctgtctcctcatcaatgctatactcaaacctataattaagcataaatttggtaatgtgatagctcttccaatagaggcagatacatgttcagcctgcacattaatcatgacatgaaagttcttgtgtactattaacagaatatagacgtcagacacaggtaggagaaatattttgaaggcagaggtctttcctggtgtccctacaatcttaccacataggctggtccctgcagtgtcgccctgcaaacctaactctacttccacggctgttccattcatacaatgtttatgggtggaacaagctttgggggaagaagggcataaggaggtggatctgcaagagagctccatggaattgggcctctgaaactgatttttgtggctctttggcctctgacagtaccactcaactgacatggtcttcactctccagagctacaagaagatatgtccatttctagctaggtaagagatgtccacctacaaccaaataaaatgggggaattaccaagagaaagcaatagaaaaatcaagtctaagagttactagtttgccttgaacttggctctagaaactggctttagaagtctagccaatcaaggctatattaaactgtgaccatgagaattagcttcaccaggtaaacttctgagcatcctttaatcctttaggacccatttcacttatgtcctcctctgagaagcattttttacttctttttttgtttgtttgtttgtgtttgtttttgtttttgtttttgagacagagtctctctctgtcacccaagctggagtgcagtggcgcaatcttggctcactgcaacctccacctcccgggttcaagcaattctcctgcctcagcctcccaagtagctgggactacaggtgcatgccaccacgcccggctaattttttgtatttttagtagagacagggtttcgcagcgttagccaggatggtcttgatctcctgacttcatgatctgcccacctcggcctcccaaagtactaggattacagatgtgagccaccgcgcccagcctgcattttttacttctttcaggcagaatttctttattccaatctagtcagccccgcagtcctttattcttagcctgttgtagcacttgtcatattgtattgtgattatttctgaatatttatgtttctatgtctagactgtagattctttgaggctgagaactatatgtcccatcatctgggtatctccagtccacagtgtgtcatacatagtgagtgcttgatgaaatatcacttgaaggaatatacatatggacattcactgggtccatgacaggatagattcgaacaagaatgttcctccaaaggccaccagactatatactaaccatgactttatgctaataatgattcatctctctgctgaaaaagtaagtggatagataggcacatggcttcttttgataaatgatatctcttaataggtaatgaagattactttctgtttggcaaatctttgtggtagagaatcatgaccaacacacgtcctaccaattttgtttagcatcaggtagtagattttttaaattatagtaattcaagctgagaatgtagatttaaaaaataaaattattgtaaattttgttttgttcttattacaaaagtcatttggggtcaatttcaaaaatatataaaagtaaacaggagaaatttaaaatgtccttcagtcccactccttcagagaaaacccctgttaatatgtaagtgcatatccttcttttttctgtgcataatactttttaaaatatttgaagtattatgcttttttaacttaaaattgtctcatgaatattttcttatgccattataatacttacctataacatcattattttttaattattcaggccctttcccgaccatgacctcatgttctctctttgtgaagtctgattacttggtgacatgatcgtgagaataagctctggcgatataagaatttcctctcttgaaggccatgctcagtaaattacttggtgacatgatcgtgagaataagctctggcgatacaagaatttcctctcttgaaggccatgctcagtaataaagttggtctcaccgaggccctgtgacaccttagaaaccacgaattgccaggctgagcaataccagtcccgcccttcccctccctggtgtttacattgagttctccttcacaatttctgcagccactccgtggccaccgtcaccttattcctgactgccacaagagtctttcaatattcctttgattgcctattccttctgaaatctaccttttcctctaatagggcaattcatcattttcaaatgcaatttttactctgatctagaacttactgtgaatccttgtcacctgccacagcaaatctaagtctagcacttaaggatcctgcagatatgctcatcgttgcttctcacttacctcattgcttagtccctctgctctaaccctgtgtgttgatcacatgtgtgtgtgtccctcttccccattagacaaaggtcttggtatgacttcagttctcttgcagggccccatcagctcttccccaaagggagctatgcagggttgactcccaatctggctttcccttatgtctcaggatctgggtggtacgtggccccttcacaaagctctgcactgagagctgaggcctcccgggcctggggtgtctgtgtctttcaggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggctttttgggagtaaaaatgtgtcttcagagactgtgaacatcaccatcactcaaggtgagacatgtgccaccctggaatgcccagggacgcctgtgtgtggaacctgcaatcacactgggaagttgagttgggaggagattcctgattcttacacgcacttcttcatatgtggttccctcctggtgatcaccaggaggtccccaaaagtccctgattgcagggtaggtttgcagctctgtttcagtccattcttttggggtagctaggaggtgtcattcactctgcagcatgatggcaggagcagaagccacatctcctccccaataaatacctctgtctttccttacgctaatcacacccacggtgtcatatgttcctatcgtgctggcctccttcttatccaagccttttagccacgatccaaactggcaggagcccctcatcccctcacagaaagagcccagaacctgggttctggccctgcagctaattaaccatctgaccagaggtgagccacttagtctctctgaaccccaatttcttcttccgtaacaaaaataagctgacatttattgggcacctttcagtgtgctagactctgtgctaaacaattctttacatgcacctggtttgactatcacagtagaccttcacaacatgagataggtaatattccattttacagatgaagtaaccgaggtgcaaaaataaataaataagtttccctaaggtcacatcaaagacttcaaagcctgtatatttaaccagtaagtaaaagatttgaacaagcactaatatcctatgatcccattaagtcatccacaaaacatctctaggttctgtagcaccagcctccagaatcagagctctagagtggtgtgcctggactttccagtttcacagaacttctatctgtaactagcccaagacataaattgtaaacaatttgcatgtagaaaggcagcaaaacaccttttgagattttgacactacaatgccataatttgtacaaaaataatttcatgacactttaaactgaaagtaaatactcccaagtggttagggaaagagagcaaataaagcaaatggggtaacatgtaaacaatgagtggatctgggtaaaggatatacgagattaaactattctggtcattttttttttaagtttggaaatatatcaaaatcaagagtttaaaaaattgaaatgcaaaatcaacaaatttgtcccagtttctagaccatagcattgtctgacaatttcttaactgtcacacaaaacccagcttacaacctaacttgttaacgctccctgtcacatctctgtcaaacaagcaggagcctttgctcagtgtttggtgagctgtcctctgctcagatagcactaagatcaggaaccaatgggaggaagcaatactttcccccagacttccccaccattcctaccacttgcctgttggctgttgtcaaagactttctactggtgacctcactgtttgttccaaatatctgccttagtgactgtcattttttttcatctctccacttctcctaataggtttggcagtgtcaaccatctcatcattctttccacctgggtaccaagtctctttctgcttggtgatggtactcctttttgcagtggacacaggactatatttctctgtgaagacaaacattcgaagctcaacaagagactggaaggaccataaatttaaatggagaaaggaccctcaagacaaatgacccccatcccatgggggtaataagagcagtagcagcagcatctctgaacatttctctggatttgcaaccccatcatcctcaggcctctctacaagcagcaggaaacatagaactcagagccagatcccttatccaactctcgacttttccttggtctccagtggaagggaaaagcccatgatcttcaagcagggaagccccagtgagtagctgcattcctagaaattgaagtttcagagctacacaaacactttttctgtcccaaccgttccctcacagcaaagcaacaatacaggctagggatggtaatcctttaaacatacaaaaattgctcgtgttataaattacccagtttagaggggaaaaaaaaacaattattcctaaataaatggataagtagaattaatggttgaggcaggaccatacagagtgtgggaactgctggggatctagggaattcagtgggaccaatgaaagcatggctgagaaatagcaggtagtccaggatagtctaagggaggtgttcccatctgagcccagagataagggtgtcttcctagaacattagccgtagtggaattaacaggaaatcatgagggtgacgtagaattgagtcttccaggggactctatcagaactggaccatctccaagtatataacgatgagtcctcttaatgctaggagtagaaaatggtcctaggaaggggactgaggattgcggtggggggtggggtggaaaagaaagtacagaacaaaccctgtgtcactgtcccaagttgctaagtgaacagaactatctcagcatcagaatgagaaagcctgagaagaaagaaccaaccacaagcacacaggaaggaaagcgcaggaggtgaaaatgctttcttggccagggtagtaagaattagaggttaatgcagggactgtaaaaccaccttttctgcttcaatatctaattcctgtgtagctttgttcattgcatttattaaacaaatgttgtataaccaatactaaatgtactactgagcttcgctgagttaagttatgaaactttcaaatccttcatcatgtcagttccaatgaggtggggatggagaagacaattgttgcttatgaaagaaagctttagctgtctctgttttgtaagctttaagcgcaacatttcttggttccaataaagcattttacaagatcttgcatgctactcttagatagaagatgggaaaaccatggtaataaaatatgaatgataaaattctttcttcttccctttgtccaacattgtaacagagattggtttggattggtaagaaacaccccctcctcccagcaaccatctcaccacaactcatataaattagccagcttgctttccaaatcttgctgagacaattgggctaaggaggattcttatgggaagtatgggataggagggtgaataagcattagagatcgttttagagcattggggcagataggagaaggcacagctacacaggaggtagaggcctgggcagaggtagagggtcagcctgattgtatgaattatgagctatataccaagacgattcaagctagattgcatacataaatattacataagattccgacacgacacaggtgcatttggaaaccttggacattcaactcacatttatttactacctacaatgtgcaagcttgagttcaggtgctgaagataccagatgaacaacacagggtcattccctggagaagctttatttctagtgagaaaaacagttaaataggaagagaatgaagaaagggctgcagaaaagaggcttgatttggggggtgtggtcatgaaggatgagtaggagttcgccaggcaaagaagagaagaaaagcccaaggttcataggcaaagattcaaaaaccagagtgtgagttcaagaaagcagtttggttctgtgtcggtgagggagaggaaagagtttcagggccagatcatgaagggcattaccttccaaactaaggagatcgtatcagaccctgcaatacattgagagagtttaagcagaccaggtttgtaccgtatagtattttagaaggattctctcgcaactacttgatggatggacgggacaggagagttgaagaccagaagccaaatagggcagcaaggcaggatgcagtaacccaaagggagcaatgaggaagtaactggcggtgaggctggagaggaaggtgcttaatcaacaaggtatttaggaggccgactctccaagaattggcagccagcagtacacggcgtgactaaggaccaggttccacacatagtgcccgttttctgagttaggaaatagaaaggcaaggcaggtacaggtttggtggaaagacaaacaattcgttttggtattattagtacttacttcctttggtcagtaaattttcttaaagtgtcagtttccataacgtaattgccgtggttaagcagctaagagttatcactacaaccctagtcggaaaaaccaaatacctcaaaattacccgtacagcactaaggcagaagaggacattgggaaccacacaacgcggaggtctgctaccagagctccctgcggttagcaccgcggctggttttgagcgccaaggccccagcgctcccagcggatagcatcgcacgcagttttttcagtcaaagtttcaaaaacccagggttcacaaaatgcgacttccgtccctgggtgggatcgaaccaccaacctttcggttaacagccgaacgcgctaaccgattgcgccacagagacgggcgttggcgattttggctgccaagtcacttcactgaagaaaaaatgctcagcactcacgtctccaaaaaaattgaggttgatttgaaaccagtgacacaattagctttccgtgcttcagggcgcggctcatagccctgagcgaggcaggtcttttttctgcgctagcacttgcctagatctggagcaggactcagcttccagcagaagaggttgagaaaaggagagcagaagagaatgcaggaacgaagggtcttcggggaatccaaaatggatgctctctgtgggttcgggggttccgttgattttggtcagagaagtacgacgataagctttttttgctgatgtagacaacttatgtatgcatgtgcacacgtttagtgctgactcataataagcttattatcgtgagcattaaaaatattttctttcaggtccaatcacgtccagcaaaatgtgatgtctaagtaagtgagttttgtgttacaaaattagtcttcaacccacgctgttttgaaaggtttctaccggcatattagacatgcagacagaacacggagcttaaaaagcctgtaacattccaattaatggtattcagcttggaaataaaaaatattttttaaaaaatgcgtgcaacttaaggactttcatgctgacatatccagatccaaatatctgaggacagagacccctaattccaccaccatcgacctagggaacctcgtcagtgctgggtctaaaaaggctttttttttttctttaattcatatgtatatatactttattcatatatatatatactttaagttctaggctacatgtgcacaaggtgcaggttcgttacatatacatgtgccatgttggtgtgctgcacccattaactcgtcatctacattaggtgtttctcctaatgttatccctccctccttcccccacccacgacaggtcccggtgtgtgatgttccctaccatgcacacgtatgtttattgtggaactattcacaatagcaaagacttggaaccaacccaaatgtccatcaatgatagactggattaagaaaatgtggcacatatacactatggaatactatgcagccataaaaaaggatgagttcatgtcctttgtagggacatagatgaagctggaaaccatcagtctgaacaaactatcacaaggacagaaaatcagacaccgcatgttctcactcataggtgggaattgaacaataagaacacttggacgaaagccattttctatattgcccaaaaaccagggtctctccatagcctccacacagaatctcctttctttctgccctgccatcctctgtcatcagtgggctccagtttaggagcaggtggaagttttcaatgatgttcagtgaaatgagaagacatgcaaacatagatatgtatatgcagaaattatatatgcatatatgtttatatgtacacagtatcatatgtataataaataagtaaataaataaataaatttgccaaatgatctttaaactagagtcatttattttttttattaatttttttttttgagatggagtcttgctctgtcgcccaggctggagtgcagtggcgcaatcttggctcactgcaacctccacctcccacattcaagcaattctcctgtctcagcctcctgagtagctgagattacagtcatgggccaccatgcccggctaatttttgtatttttttttttttttttttgagacagagtctcgctgtcgcccaggttggagtgcagtggcgcgatctcggctcactgcaggctccgccccccgggattcacgccattctcctgcctcagcctcctgagtagctgggactacaggcacctgccacctcgcccggctaatttttttgtatttttggtagagatgggctttcgctatgttggccaggctggtctcaaactcctgacctcaggtgatcctcctgcctcagcctcccaaactgctgggattacaggtgagagccactgtaccaggcctagagtcatttcttttatactttaaatttttgtctctgttcttttgctcagacctgtggagctggcaatatgggcaagtgtcatggactgtctactgccaggaagctccattgtcaccgacaggatcagaagtggcatggtaaatggtacaagaaagcccattcgggcacagtcctgaagaccagcctttttggaggtgcttctcatgcaaagggaattgggctggaaaaagtagggattggagccaaatagcccagctctgccactgagaagtgtgccagggccaagctgatcatccagcataagctagatgctgtggtctccactggcacagctgatcctcttgttacaggatggaggctgtgaggcagatgagagaacagcaagaaaatcacagcctttgtacctgatgatgattgcttgaattttattgaaaaaaatgatgaagttctgtatcaggggaaccagcacccaatatttcaatgtaggttct

(19) United States
(12) Patent Application Publication
(10) Pub. No.: US 2007/0231813 A1
(43) Pub. Date: Oct. 4, 2007

(54) Fcgr3a Gebotype and Methods for Evaluating Treatment Response to Non-Depleting Antibodies

(75) Inventors: Guillaume Cartron, Savonnieres (FR); Herve Watier, Ballan-Mire (FR); Philippe Colombat, Larcay (FR)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: Centre Hospitalier Regional et Universitaire de Tours, Tours Cedex (FR)

(21) Appl. No.: 11/597,981

(22) PCT Filed: May 31, 2005
(86) PCT No.: PCT/IB05/02310

§ 371 Date: Nov. 30, 2006

Related U.S. Application Data
(60) Provisional application No. 60/575,411, filed on Jun. 1, 2004.

(51) Int. Cl. C12Q 1/68; G01N 33/53

(52) U.S. Cl. 435/6; 435/7.1; 435/7.24

(57) ABSTRACT

The present invention relates to methods and compositions to evaluate or assess the response and/or side effects of a subject to particular therapeutic treatment. More particularly, the invention provides methods to determine the response and/or side effects of subjects, or to adapt the treatment protocol of subjects treated with therapeutic antibodies in situations where target neutralisation is desired without depletion of a target cell. The invention is based on a determination of the FCGR3A genotype of a subject. Preferable, the therapeutic antibodies are antibodies or proteins comprising Fc portions of the G4 subclass.

The present invention relates to methods and compositions to evaluate or assess the response and/or side effects of a subject to particular therapeutic treatment. More particularly, the invention provides methods to determine the response and/or the side effects of subjects, or to adapt the treatment protocol of subjects treated with therapeutic antibodies. The invention can be used for patients treated with antibodies designed to target a membrane antigen but which need not deplete target cells, and is suited to identify subjects having increased or decreased threrapeutic response to the therapy, or to identify subjects having increased or decreased risk of side effects, particularly undesired side effects such as cytokine release syndrome.

### INTRODUCTION

Various therapeutic strategies in human beings are based on the use of therapeutic antibodies which need not be depleting to a cell to which the antibody is bound. Some such strategies aim to use antibodies to antagonize or agonize membrane receptors and/or to target membrane antigens with the aim of modulatating cell function. A wide range of therapeutic modalities are encompassed by such strategies, including but not limited to strategies to modulate immune cell function (e.g. B cells, T cells, NK cells), strategies to treat inflammatory conditions and/or autoimmune disorders, anti-tumor strategies (for example, anti-erbB1 and erbB2 antibodies such as trastuzumab, pertuzumab and ABX-EGF; anti-TRAIL-R antibodies, anti-CD33 antibody Mylotarg®), and treatments for heart disease or anti-thrombosis (e.g. ReoPro®). In almost all these applications, the antibody appears to act by antagonizing a key membrane receptor or to induce intra-cellular signaling. Such antibodies are typically monoclonal antibodies, of IgG species, typically IgG4, and most preferably will be full-length antibodies so as to have a longer in vivo half-life. They are often developed in the treatment of inflammatory disorders such as psoriasis, multiple sclerosis, rheumatoid arthritis, Crohn's disease but can also be used in cancer, for example, when Fc-mediated ADCC mechanism is not required (see Mylotarg, ABX-EGF which targets tumor cells, for example, or an anti-NKp30 antibody which activates an NK cel). The preferential use of IgG4 prevents cytolysis but maintains a long in vivo half-life due to interactions with FcRn receptors. This immunoglobulin subclass is therefore generally used when the recruitment of effectors are not needed A particular example of such therapeutic antibodies is CDP571 (also referred to as Humicade™, which is an anti-TNFα IgG4 monoclonal antibody. Other examples of IgG4 antibodies include clenoliximab, which is used in the treatment of Rheumatoid Arthritis or ch5D12, which is used in the treatment of Multiple sclerosis, aselizumab (HuDreg55), an anti-selectin L antibody of the IgG4 type, cedelizumab OKTcdr4a, humanized (anti-CD4) and rovelizumab Hu23F2G (humanized (anti-CD11a). Yet other examples of antibodies not required to have target cell depleting activities are antibodies that modulate NK cells by targeting NK cell surface receptors, for example anti-NKG2D, anti-NKp30, anti-NKp46 or anti-NKp44 or anti-CD94/NKG2A antibodies. It is nevertheless possible to obtain IgG1 antibodies which are not capable of inducing cytolytic activity, for example Mason U, et al. a Rheumatol. 2002; 29(2): 220-9) provide an example where an anti-CD4 antibody of the IgG1 type has efficacy only if it does not substantially induce lysis. The latter demonstrates that certain IgG1 antibodies can be free of depleting activity, but highlights the risks in developing of an IgG1-based product for such an indication, particularly as lytic activity may depend on glycosylation state.

The antibodies can be recombinant antibodies (chimeric, humanized, primatized, “fully” human, or modified in any way, including but not limited to modifications that reduce binding to FcγRIIIa) comprising functional domains from various species or origin or specificity. Also envisioned are fusion proteins comprising an Fc portion.

While such non-depleting antibodies represent a novel and target-specific approach to human therapy, they do not always exhibit strong efficacy and often show unwanted side effects, such that their use could be improved by evaluating the nature of the response of subjects thereto. For example, one antibody which does not require target cell depletion for efficacy has nevertheless been suggested to be depleting in one patient (saacs J D, Wing M G, Greenwood J D, Hazleman B L, Hale G, Waldmann R A therapeutic human IgG4 monoclonal antibody that depletes target cells in humans. Clin Exp Immunol. 1996 December; 106(3):427-33). This target depleting effect may diminish efficacy or the product in applications where depleting is unwanted; predicting such a depletion effect could therefore have an important therapeutic value, as such a patient could be treated with a different product or according to a different regimen.

In another example, it is known that depleting antibodies often lead to cytokine-release syndrome, including severe cytokine-release syndrome,. Predicting such an effect in patients to be treated with antibodies which are not required or not intended to be target depleting would be useful in therapy. Likewise, predicting such an effect in patients to be treated with depleting antibodies would be great value as well, for example in order to modify the therapeutic regimen by providing the patient with premedication effective to reduce or prevent cytokine release syndrome. Examples of depleting antibodies suggested to induce cytokine release syndrome include the CAMPATH 1-H monoclonal antibody (Wing et al, 1996, J Clin Invest 98, 2819-2826) and the anti-CD20 antibody Mabthera (rituxiab), where symptoms presumed to be due to cytokine release syndrome included fever and chills, flushing, angiodema, nausea, urticaria/rash, hypotension and bronchiospasm (Committee for Proprietary Med. Prod. Europ. Public Assessment Report, CPMP/259/98, The Europ. Agency for the Evaluation of Med Prod. (1998)).

The availability of methods allowing the evaluation of patient response and/or side effects to antibody treatment would greatly enhance the therapeutic efficacy of therapies since benefit/risk ratio of treatment can be better evalutated, treatments can be adjusted, and finally novel therapeutic products can be developed.

### SUMMARY OF THE INVENTION

The present invention now proposes novel methods and compositions to assess the therapeutic response of a subject to a therapeutic composition comprising an Fc portion, preferably a therapeutic antibody. Preferably, said therapeutic antibody is an antibody which is not capable of, or is not required to be capable of, depleting target cells, more preferably an antibody which is not capable of, or which is not or is not required to be capable of inducing ADCC-mediated depletion of target cells. In preferred examples, the antibody is an IgG4 antibody. In other examples the antibody is an antibody other than an IgG4 which is not capable of, or which is not or is not required to be capable of inducing ADCC- or ADCP-mediated depletion of target cells. The antibody may also be an antibody other than an IgG4 which does not substantially induce target depletion, or which preferably does not induce target depletion in an individual having a phenylalanine at position 158 of the FcγRIIIa receptor. Preferably such a non-IgG4 antibody comprises one or more modifications in an Fc region so as to reduce binding to FcγRIIIa.

Preferably, the therapeutic response of the subject relates to efficacy in the treatment of a disorder where a membrane antigen is targeted by an antibody, but where depletion of a target cell, particularly via an ADCC- or ADCP-mechanism, is not required. Preferred examples of such disorders to be treated include but are not limited to infectious disesase, immune disorders such as inflammatory disorders and autoimmune disorders, transplantation, cancer, and cardiovascular disorders. Disorders may also generally include any disorder in which an immune response is either to be augmented or inhibited. For example, antibodies capable of activating NK cells (e.g. anti-NCR or anti-KIR) or T cells (for example anti-CTLA4), but which do not require, and moreover seek to avoid, lysis of the targeted NK or T cell, can be used in the treatment or prevention of cancer or infectious disease. In other examples, antibodies targeting and antagonizing membrane antigens erbB1 et erbB2 (trastuzumab, pertuzumab, cetuxinab, ABX-EGF) act via an anti-proliferative or anti-antiogenic activity are therefore do not require target cell lysis, particularly lysis via an ADCC mechanism. In further examples, anti-VEGFR antibodies may be sought which inhibit the VEGF receptor but which do not induce lysis of the target cell so as to avoid any potential damage to the endothelium, to avoid inflammation, and to avoid potential thrombosis. Preferably, a therapeutic response of a subject relates to modulation of a physiological pathway, more preferably an anti-inflammatory effect, an anti-proliferative effect, an inhibition or a potentiation of an immune response.

The present invention further proposes novel methods and compositions to assess the side effects of a subject to a therapeutic antibody which is intended to be non-depleting to a cell to which it is bound, or more particularly intended to avoid lysis of an antibody coated cell by ADCC- or ADCP-. Side effects can be the consequence of the induction of cytokine release or of unwanted ADCC- or ADCP- activity. The invention also proposes methods to select patients having best responding profile to therapeutic antibody treatment. The invention also relates to methods of treating patients with therapeutic antibodies, comprising a prior step of evaluating the patient's response. The invention also relates to compositions and kits suitable to perform the invention. The invention may as well be used in clinical trials or experimental settings, to assess or monitor a subject's response, or to verify the mode of action of an antibody. Three classes of FcγR (FcγRI, FcγRII and FcγRIII) and their subclasses are encoded by eight genes in humans, all located on the long arm of chromosome 1. Some of these genes display a functional allelic polymorphism generating allotypes with different receptor properties. One of these genetic factors is a gene dimorphism in FCGR3A, which encodes FcγRIIIa with either a phenylalanine (F) or a valine (V) at amino-acid position 158 of the mature protein (Koene et al, Blood. 1997;90:1109-1114; Wu J, et al. J Clin Invest. 1997; 100: 1059-1070). It has been demonstrated that human IgG4 binds more strongly to homozygous FcγRIIIa-158V natural killer cells (NK) than to homozygous FcγRIIIa-158F or heterozygous NK cells (Koene et al, 1997).

The present invention provides that a correlation between the genotype of a subject and its ability to respond to treatment with a composition comprising an Fc portion, particularly an antibody, can be used to predict the subject's response and side effect sensibility to treatment with a such therapeutic composition which is intended to be substantially non-depleting. More specifically, the invention provides that the genotype of the FcγRIIIa receptor can be used to predict a subject's response and side effect sensibility to treatment a composition comprising an Fc portion, particularly an antibody, which is intended to be non-depleting. As further described herein, it will be appreciated that the genotype of a patient can be determined by any suitable manner, including by determining the nucleotide sequence present in the DNA of a patient or by determining the amino acid present in the FcγRIIIa receptor of a patient, the latter for example according to standard approaches for determining the phenotype or allotype of a patient at the receptor.

More particularly, the present invention provides that the presence in a subject of a Valine at position 158 of the FcγRIIIa receptor is indicative of a decreased response to said treatment in terns of efficacy, and a phenylalanine at position 158 of the FcγRIIIa receptor is indicative of an increased response to said treatment. Moreover, the present invention provides that the presence in a subject of a Valine at position 158 of the FcγRIIIa receptor is indicative of a increased side effects or susceptibility to side effects to said treatment and a phenylalanine at position 158 of the FcγRIIIa receptor is indicative of decreased side effects or susceptibility to side effects to said treatment.

The identity of the amino acid residue present at position 158 of the FcγRIIIa receptor corresponds to a nucleotide polymorphism at nucleotide at position 559 of the FCGR3A cDNA or 4985 of the FCGR3A genomic DNA. Thus, determining the genotype of a subject can readily be carried out by determining the nucleotide present in the DNA of a subject or by determining the amino acid residue present in the FcγRIIIa receptor.

Side effects can be the consequence of the induction of cytokine release or of activation of FcγrIIIa-bearing cells, particularly ADCC- or ADCP- mediated by NK cells and/or macrophages. Examples of cytokines released are TNF-α, IFN-γand IL-6. Symptoms of cytokine release often include but are not limited to fever and chills, flushing, angiodema, nausea, urticaria/rash, hypotension and bronchiospasm.

Accordingly, the present invention provides a method based on an association between the FCGR3A genotype and clinical and molecular responses to a composition comprising an Fc portion, particularly an antibody, intended to be non-depleting, preferably which is not intended to induce cytolytic activity, particularly ADCC or ADCP, of a cell with which it is associated or bound. The invention thus provides a marker that can be used to monitor, evaluate or select a patient's response to such antibodies. This invention thus introduces pharmacogenetic approaches in the management of patients with therapeutic antibody treatment, more particularly a therapeutic treatment by a substantially non-depleting antibody, especially an IgG4 antibody. In one aspect, the substantially non-depleting antibody is an antibody which binds to a receptor present on the surface of an immune cell, more preferably a B cell, a T cell or a NK cell. In another preferred aspect, the substantially non-depleting antibody is an antibody which binds to a receptor present on the surface of a tumoral cell or an endothelial cell; examples of receptors include but are not limited to erbB1 and erbB2 on tumor cells, and cellular VEGF-R on endothelial cells. Other examples or receptors are known in the art and/or, described in Table 1, and further described herein.

It will be appreciated that the methods of the invention can be practiced with a wide range of Fc portion-containing compositions, especially therapeutic antibodies. In general, the antibodies or Fc portion-containing compositions are intended to be non-depleting, preferably which do not substantially induce or are intended not to induce cytolytic activity, particularly ADCC or ADCP activity, toward a cell with which they are associated or bound. For example, the therapeutic antibody can be of an IgG4 subtype. Preferably, said therapeutic antibody specifically binds a membrane antigen on a target cell, for example including but not limited to a tumor cell, an endothelial cell, a B lymphocyte, a T lymphocyte or and NK cell. In preferred examples, said therapeutic antibody binds and activates an NK cell activatory receptor. In another example, said therapeutic antibody binds and interferes with ligand binding to or inhibits the function of an NK cell activatory receptor. In another example, said therapeutic antibody binds and interferes with ligand binding to or inhibits the function of an NK cell inhibitory receptor, thereby activating or potentiating the activation of an NK cell. In another example, said therapeutic antibody binds and activates an NK cell inhibitory receptor, thereby inhibiting the activation of an NK cell. In another aspect, said composition comprising an Fc portion, particularly an antibody, specifically binds a membrane pro-inflammatory cytokine or a lymphocyte receptor.

In a preferred embodiment, a subject to be treated according to the invention has a disorder such as a tumor. In another example a subject has another proliferative disease, for example a hyperproliferative conditions such as hyperplasias, fibrosis (especially pulmonary, but also other types of fibrosis, such as renal fibrosis), angiogenesis, psoriasis, atherosclerosis and smooth muscle proliferation in the blood vessels, such as stenosis or restenosis following angioplasty. In another example a subject has an inflammatory or preferably autoimmune disorder, examples of such disorders include but are not limited to disorders mediated by phagocytic cells, which includes macrophages and neutrophil granulocytes (Polymorphonuclear leukocytes, PMNs) and/or T cells. Examples include inflammatory skin diseases including psoriasis; responses associated with inflammatory bowel disease (such as Crohn's disease and ulcerative colitis); adult respiratory distress syndrome; dermatitis; CNS inflammatory disorders such as multiple sclerosis; uveitic disorders; allergic conditions such as eczema and asthma and other conditions involving infiltration of T cells and chronic inflammatory responses; skin hypersensitivity reactions (including poison ivy and poison oak); autoimmune diseases such as rheumatoid arthritis, systemic lupus erythematosus (SLE), diabetes mellitus, multiple sclerosis, Raynaud's syndrome, autoimmune thyroiditis, Sjogren's syndrome, juvenile onset diabetes, and immune responses associated with delayed hypersensitivity mediated by cytokines and T-lymphocytes typically found in tuberculosis, sarcoidosis, polymyositis, granulomatosis and vasculitis; pernicious anemia; multiple organ injury syndrome secondary to septicaemia or trauma; autoimmune haemolytic anemia; myethemia gravis; antigen-antibody complex mediated diseases; all types of transplantation rejection, including graft vs. host or host vs. graft disease.

It will be appreciated that any embodiments of the invention relating to a therapeutic antibody can relate to a complete antibody or a fragment or modified antibody, so long as the antibody comprises an Fc portion. As further discussed herein, there are five types of human immunoglobulin Fc regions with different effector and pharmacokinetic properties: IgG, IgA, IgM, IgD, and IgE. IgG is the most abundant immunoglobulin in serum. IgG also has the longest half-life in serum of any immunoglobulin (23 days). Unlike other immunoglobulins, IgG is efficiently recirculated following binding to a particular Fc receptor called FcRn which is structurally and genetically unrelated to FcγR. There are four IgG subclasses G1, G2, G3, and G4, each of which have different effector functions. G1 and G3 can bind C1q and activate complement by the classical pathway while G2 and G4 cannot. Even though G3 is able to bind C1q more efficiently than G1, G1 is more effective at mediating complement-directed cell lysis. The C1q binding site in IgG is located at the carboxy terminal region of the CH2 domain. All IgG subclasses are capable of binding to Fc receptors (CD16, CD32, CD64) with G1 and G3 being more effective than G2 and G4. The Fc receptor-binding region of IgG is formed by residues located in both the hinge and the carboxy terminal regions of the CH2 domain. IgA can exist both in a monomeric and dimeric form held together by a J-chain. IgA is the second most abundant Ig in serum, but its half-life is only 6 days. IgA has three effector functions. It binds to an IgA specific receptor on macrophages and eosinophils, associated with phagocytosis and degranulation respectively, and resulting in antibacterial activity and especially cytotoxicity, for example towards parasites. It can also fix complement via the alternative pathway. In particularly preferred embodiments, the invention is carried out in accordance with an Fc region of the G4 subclass (also referred to as IgG4 herein and γ4 isotype when referring to the entire set of constant regions on the heavy chain).

Any protein comprising an Fc portion of an immunoglobulin, an analog of the Fc portion of an immunoglobulin, or a fragment of the Fc portion of an immunoglobulin can be used in accordance with the invention. In particularly preferred embodiments, such proteins comprise a protein of interest (e.g. other than an Fc portion of an Ig) fused to an Fc portion of an immunoglobulin. The protein of interest may be fused directly, or fused via a peptide linker, to the Fc portion. The Fc portion may be fused to the protein of interest at either terminus or at both termini. These heterologous fusion proteins are known to have biologically activity and have an increased half-life compared to native protein of interest. One example is a TNF-alpha receptor protein fused to an Fc portion, for example etanercept (ENBREL®, Amgen, USA). Another example is alfacept (Amevive), a fusion protein comprising an Fc region of the IgG1 subtype fused to the first extracellular domain of human LFA-3, or preferably a modified Fc(IgG1)-LFA-3 or other Fc-LFA-3 fusion protein which is substantially non-depleting or which has diminished depleting activity. Preferably, as discussed in relation to therapeutic antibodies, said fusion protein will preferably be intended to be non-depleting, preferably intended not to induce cytolytic activity, particularly ADCC or ADCP, of a cell with which it is associated or bound. ADCC or ADCP, when referring to the activity mediated by a fusion protein can also interchangeably be referred to as fusion protein-dependent cell-mediated cytotoxicity (FPDCC) and fusion protein-dependent cell-mediated phagocytosis (FPDCP). Preferably the Fc portion will be of a subtype having relatively low binding to the FcγRIIIa receptor or will be a modified so as to decrease binding to the FcγRIIIa receptor.

An additional object of this invention resides in a method of assessing the response of a subject to a treatment with a protein comprising an Fc portion of the IgG4 subtype, comprising determining amino acid residue at position 158 of FcγRIIIa receptor, a Valine at position 158 being indicative of a lower response to said treatment and a phenylalanine at position 158 being indicative of a better response to said treatment.

A further object of this invention is a method of selecting patients for treatment with an Fc portion-containing composition, preferably an antibody, intended to be non-depleting, preferably which is intended not to induce cytolytic activity, particularly ADCC or ADCP, of a cell with which it is associated or bound, the method comprising determining amino acid residue at position 158 of FcγRIIIa receptor, and selecting the patients having a phenylalanine at position 158 for said treatment. Said method can also be carried out by determining the nucleotide present at nucleotide position 559 of the FCG3A cDNA or nucleotide position 4985 of the FCG3A gene. Preferably, said therapeutic antibody is an IgG4.

An other object of this invention is a method of assessing the side effects of a subject to a treatment with a composition comprising an Fc portion, preferably an antibody, intended to be non-depleting, preferably which is intended not to induce cytolytic activity, particularly ADCC or ADCP, of a cell with which it is associated or bound, comprising determining amino acid residue at position 158 of FcγRIIIa receptor, a Valine at position 158 being indicative of a higher side effects to said treatment and a phenylalanine at position 158 being indicative of a lower side effects to said treatment. Again, said method can also be carried out by determining the nucleotide present at nucleotide position 559 of the FCG3A cDNA or nucleotide position 4985 of the FCG3A gene.

The methods of the invention can be used particularly advantageously in methods of treatment of disease. Preferably, said FcγRIIIa receptor genotype is indicative of the consequences of said therapy. In one example, the methods of the invention are used to determine the amount and administration regimen of a therapeutic composition to be administered to a subject. In another example, the methods of the invention are used to select a therapeutic composition to be administered to a subject—for example therapeutic compositions that are less likely to induce ADCC or ADCP of an antibody-bound cell or less likely to induce cytokine release by immune cells.

If a subject is determined to have a Valine at position 158 of the FcγRIIIa receptor, this subject is deemed to have increased susceptibility to side effects and/or decreased response to treatment (eg. the treatment is less efficacious). Such a subject may be more advantageously treated with a composition or according to a regimen likely to provide increased efficacy in such a subject. An example of a composition that could be more suitable for such a patient is an antibody compound having decreased ADCC or ADCP activity, or decreased binding to the FcγRIIIa receptor compared to another antibody which specifically binds the same biological target protein. In one aspect, binding of an antibody to the FcγRIIIa receptor can be assessed using the methods of Koene et al, Blood (1997) or using the BIAcore system (Okasaki A et al. J Mol Biol 2004, 336: 1239-1249). Alternatively, an antibody is administered to a subject who has a Valine at position 158 of the said FcγRIIIa receptor at different doses or administration schedules, preferably lower doses, with conjoint treatment (such as premedication to treat or prevent cytokine release syndrome) than a subject having a phenylalanine at position 158. Preferably said administration schedule, dosing or selection of compound is intended to result in decreased lysis of antibody-bound cells and/or decreased cytokine release. Antibodies having decreased ability to bind the FcγRIIIa receptor are known and can be prepared according to methods. In certain embodiments, an antibody will contain an amino acid modification to diminish the interaction with Fc receptors, e.g. by altering certain residues within the Fc region that are known to diminish the interaction with Fc receptors, or, using well known methods, to experimentally identify amino acid alterations within the Fc region (or elsewhere within the Ig) that can reduce the binding to Fc receptors. In the case of Fc regions from the IgG1 subtype in antibodies where depletion is not desired, modifications can be carried out as described in Shields et al. (J. Biol. Chem. 276:6591-6604); PCT Publication No. WO 2004/126750), the disclosures of which are incorporated herein by reference.

The invention also provides a method for monitoring or treating a subject, the method comprising:

determining the FCGR3A genotype in the subject, wherein the genotype is correlated with an increased or decreased likelihood of responding to a treatment with an Fc portion-containing composition, particularly an antibody, or with an increased or decreased likelihood of having a side effect as a result of said antibody treatment; and

monitoring said subject for the development of a side effect to treatment or for efficacy of the antibody treatment.

The invention further provides a method for treating a subject, the method comprising:

determining FCGR3A genotype in the subject, wherein the genotype is correlated with an increased or decreased response to treatment with an Fc portion-containing composition, or with increased or decreased likelihood of exhibiting side effects as a result of said antibody treatment; and
selecting or determining a suitable therapy for treatment of the subject. Preferably the step of selecting or determining a suitable therapy for treatment of the subject comprises selecting a composition to be administered to the subject. In other aspects, the step of or determining a suitable therapy for treatment of the subject comprises selecting a dosage, frequency of administration or duration of treatment with a therapeutic composition to administer to the subject. Preferably the method further comprises (c) administering a therapy, preferably a composition, selected in step (b) to the subject. Preferably, the composition to be administered to a subject is an antibody composition having decreased binding to a FcγRIIIa polypeptide, preferably to an FcγRIIIa polypeptide comprising a Valine at position 158.

In another aspect, when a subject is determined to have an increased susceptibility to developing side effects upon treatment with an Fc portion-containing composition, particularly an antibody (e.g. cytokine release syndrome) or when a subject is determined to have decreased response (efficacy to treatment with an Fc portion-containing composition, particularly an antibody, the patient can be administered a treatment intended to either reduce said side effects or to improve the efficacy of the treatment, for example to inhibit ADCC or ADCP mediated by NK and/or macrophages.

Test-Composition Assessment and Clinical Trials

The methods of the invention can also be used advantageously in a clinical trial to assess subjects' therapeutic response to treatment with an Fc portion-containing composition, particularly an antibody, or susceptibility to side effects induced by such treatment. Such methods are expected to be useful for example to determine whether one, two or more arms of a clinical trial are balanced for the number of subjects having increased or decreased response to the treatment. The method of the invention can also be used to select subjects for inclusion in a clinical trial. Methods of the invention are expected to be especially useful in clinical trials where efficacy or side effects of a test composition are to be assessed.

Use of a method of the invention in a clinical trial is particularly suitable where the test composition, is known to or suspected of being capable of inducing an antibody-mediated side effect, particularly an NK cell or macrophage- mediated side effect such as cytokine release syndrome, or wherein the test composition is suspected to bind, via its Fc region to a FcγRIIIa polypeptide, preferably to an FcγRIIIa polypeptide comprising a valine at position 158.

For instance, the methods of the invention can comprise determining in a subject the FCGR3A genotype, wherein said genotype places said subject into a subgroup for treatment or analysis in a clinical trial, or in a subgroup for inclusion in a clinical trial.

In one aspect, the invention provides a method for the clinical testing of a test composition, the method comprising the following steps.

(a) administering a test composition to a plurality of individuals; and

(b) identifying one or a plurality of individuals having a first FCGR3A genotype and one or a plurality of individuals having a second FCGR3A genotype, preferably wherein a first FCGR3A genotype indicates increased susceptibility to side effects or decreased response to treatment with an Fc portion-containing composition, particularly an antibody, a second FCGR3A genotype indicates decreased susceptibility to side effects or decreased response to treatment with an Fc portion-containing composition, particularly an antibody. The method may optionally further comprise: (a) assessing the response to said test composition in said individual(s) having a first FCGR3A genotype; and/or (b) assessing the response to said test composition in the individuals having a second FCGR3A genotype. Preferably, the response to said test composition is assessed both in individuals having said first and said second FCGR3A genotype. Preferably said response is assessed separately in said first and second subpopulations of individuals. Assessing the response to said test composition preferably comprises assessing therapeutic efficacy of the test composition.

The invention also concerns a method for the clinical testing of a test composition, the method comprising the following steps.

identifying a first population of individuals having a first FCGR3A genotype and a second population of individuals having a second FGGR3A genotype;

administering a test composition to individuals of said first and/or said second population of individuals. In one embodiment, the test composition is administered to individuals of said first population but not to individuals of said second population. In one embodiment, the test composition is administered to individuals of said second population but not to individuals of said first population. In another embodiment, the test composition is administered to the individuals of both said first and said second populations. Preferably the test composition is an Fc portion-containing composition, particularly an antibody.

In the methods of monitoring or treating a subject, and in methods of clinical testing described herein, the Fc portion-containing composition is preferably intended to be non-depleting, preferably which is intended not to induce cytolytic activity, particularly ADCc or ADCP, of a cell with which it is associated or bound. Most preferably the Fc portion-containing composition comprises an Fc region of subclass G4.

### DESCRIPTION OF THE FIGURES

FIG. 1: Genetic organization of the human FCGR3A gene

FIG. 2: Amino acid sequences of human FcγRIIIa-158F (SEQ ID NO:7)

FIG. 3: Nucleic acid sequence of human FCGR3A -158F (SEQ ID NO:8)

### DETAILED DESCRIPTION

As used herein, the following terms have the meanings ascribed to them unless specified otherwise.

The term “antibody,” as used herein, refers to polyclonal and monoclonal antibodies, as well as fragments and derivatives thereof. Depending on the type of constant domain in the heavy chains, antibodies are assigned to one of five major classes: IgA, IgD, IgE, IgG, and IgM Several of these are further divided into subclasses or isotypes, such as IgG1, IgG2, IgG3, IgG4, and the like. An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids that is primarily responsible for antigen recognition. The terms variable light chain (V) and variable heavy chain (V refer to these light and heavy chains respectively. The heavy-chain constant domains that correspond to the different classes of immunoglobulins are termed “alpha,” “delta,” “epsilon,” “gamma” and “mu,” respectively. The subunit structures and three-dimensional configurations of different classes of immunoglobulins are well known. IgG and/or IgM are the preferred classes of antibodies employed in this invention, with IgG being particularly preferred, because they are the most common antibodies in the physiological situation, because they are most easily made in a laboratory setting, and because IgGs are specifically recognized by Fc gamma receptors. Preferably the antibody of this invention is a monoclonal antibody. Particularly preferred are humanized, chimeric, human, or otherwise-human-suitable antibodies.

A “therapeutic antibody” refers to any antibody, derivatized antibody, or antibody fragment that can be safely used in humans for, e.g. the therapeutic methods described herein. Human-suitable antibodies include all types of humanized, chimeric, or fully human antibodies, or any antibodies in which at least a portion of the antibodies is derived from humans or otherwise modified so as to avoid the immune response that is provoked when native non-human antibodies are used.

For the purposes of the present invention, a “humanized” antibody refers to an antibody in which the constant and variable framework region of one or more human immunoglobulins is fused with the binding region, e.g. the CDR, of an animal immunoglobulin. Such humanized antibodies are designed to maintain the binding specificity of the non-human antibody from which the binding regions are derived, but to avoid an immune reaction against the non-human antibody.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. In preferred embodiments of the present invention, the chimeric antibody nevertheless maintains the Fc region of the immunoglobulin, preferably a human Fc region, thereby allowing interactions with human Fc receptors on the surface of target cells.

The term “specifically binds to” means that an antibody can bind preferably in a competitive binding assay to the binding partner, e.g. an NK receptor such as NKp30, NKp44, NKp46, NKG2D or CD94/NKG2A, a membrane antigen such as CD2, CD3, CD4, CD25, CD28, CD40, CD11/18, ICAM alpha-4-integrin or CTLA4, as assessed using either recombinant forms of the proteins, epitopes therein, or native proteins present on the surface of isolated NK or relevant target cells. Competitive binding assays and other methods for determining specific binding are further described below and are well known in the art.

By “ADCC” or “antibody dependent cell-mediated cytotoxicity” as used herein is meant the cell-mediated reaction wherein nonspecific cytotoxic cells that express FcγRs, particularly FcγRIIIa, recognize bound antibody on a target cell and subsequently cause lysis of the target cell.

By “ADCP” or antibody dependent cell-mediated phagocytosis as used herein is meant the cell-mediated reaction wherein nonspecific cytotoxic cells that express FcγRs, particularly FcγRIIIa, recognize bound antibody on a target cell and subsequently cause phagocytosis of the target cell.

By “amino acid modification” herein is meant an amino acid substitution, insertion, and/or deletion in a polypeptide sequence. The preferred amino acid modification herein is a substitution.

The terms “isolated”, “purified” or “biologically pure” refer to material that is substantially or essentially free from components which normally accompany it as found in its native state. Purity and homogeneity are typically determined using analytical chemistry techniques such as polyacrylamide gel electrophoresis or high performance liquid chromatography. A protein that is the predominant species present in a preparation is substantially purified.

The term “biological sample” as used herein includes but is not limited to a biological fluid (for example serum, lymph, blood), cell sample or tissue sample (for example bone marrow).

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

A “label” or a “detectable moiety” is a composition detectable by spectroscopic, photochemical, biochemical, immunochemical, or chemical means. For example, useful labels include ³²P, fluorescent dyes, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (nonrecombinant) form of the cell or express native genes that are otherwise abnormally expressed, under expressed or not expressed at all.

In one example, a composition comprising an Fc portion, preferably a therapeutic antibody or an Fc fusion protein, blocks or neutralizes a key cytokine or a receptor-ligand interaction in the immune system. The therapeutic activity of said antibody does not need or is not based on the NK cell cytolytic activity and the ADCC induction. The therapeutic antibodies may by polyclonal or, preferably, monoclonal. They may be produced by hybridomas or by recombinant cells engineered to express the desired variable and constant domains. Methods for producing antibodies in hybridomas are known, for example U.S. Pat. No. 6,677,138 and PCT Publication No. WO 00/58499, the disclosures of which are incorporated herein by reference. The antibodies may by single chain antibodies or other antibody derivatives retaining the antigen specificity and the lower hinge region or a variant thereof. These may be polyfunctional antibodies, recombinant antibodies, ScFv, humanized antibodies, or variants thereof. Therapeutic antibodies are specific for surface antigens, e.g., membrane antigens or for free antigens. In preferred aspects, therapeutic antibodies may be specific for membrane bound cytokines such as TNFα, or for cell surface molecules such as T-lymphocyte or B-lymphocyte cell surface molecules, or an NK receptor. Examples of membrane bound molecules include NKp30, NKp44, NKp46, NKG2D, FcγRIIIa or CD94/NKG2A, CD2, CD3, CD4, CD25, CD28, CD40, CD11/18, ICAM, alpha-4-integrin or T-lymphocyte-associated antigen-4 (also known as CTLA-4 and CD152 antigen). In another preferred embodiment, antibodies are used in treatment of transplantation rejection, including graft vs. host or host vs. graft disease, such as for example the non-depleting IgG1 anti-CD25 antibodies (such as basiliximab and daclizumab), anti-CD25 antibodies of the IgG4 type, antibodies inhibiting T-cell costimulatory receptors, anti-CD28 antibodies where T cell depletion and/or APC mediated proliferation is to be avoided. The therapeutic antibodies are preferably of the IgG4 type, or the IgG1 type and comprising an amino acid modification or so produced so as to have diminished binding to FcγRIIIa.

Typical examples of therapeutic antibodies to be used in accordance with the invention that need not be depleting toward a target cell are listed in Table 1.

TABLE 1

| | | Commercial | | |
|---|---|---|---|---|
| Ab specificity | DCI | name | IgG | Typical Indications |
| Anti-TNFα | CDP571 | Humicade ™ | 4 | Crohn's disease |
| | | | | Rheumatoid Arthritis |
| Anti-CD4 | clenoliximab | | 4 | Rheumatoid Arthritis |
| Anti-CD4 | IDEC-151 | | 4 | Rheumatoid Arthritis |
| Anti-CD40 | Ch5D12 | | 4 | Multiple sclerosis |
| Anti-CD18 | 1B4 | | 4 | Acute inflammatory disease |
| Anti-CD11/18 | Hu23F2G | | 4 | Multiple sclerosis; Stroke; |
| or anti-LFA1 | | | | Myocardial infarction |
| Anti-CD33 | gemtuzumab | Mylotarg ® | | Cancer |
| | ozogamicin, | | | |
| Anti-EGFR | ABX-EGF, | | | Cancer |
| | panitumumab | | | |
| Anti-CTLA4 | CP-675,206, | | | Cancer, infectious disease |
| | MDX-CTLA4 | | | |
| Anti-alpha-4 | natalizumab | | | Crohn's, IBD, multiple |
| integrins | | | | sclerosis |
| Anti ICAM-1 | BIRR-1 | | | Stroke |
| Anti CD3 | anti-CD3 OKT3 | Orthoclone | | Allograft rejection |
| | (IgG2a murine) | | | |
| Anti-CD25 | basiliximab, | | | Transplantation |
| | daclizumab | | | |
| Anti-CD16 | WO 03/101485 | | | Immune disorders |
| (FcγRIIIa) | | | | characterised by autoantibodies |
| Fc-LFA- | alfacept | Amevive ™ | | |
| 3(CD2- | | | | |
| binding) | | | | |
| fusion protein | | | | |

Within the context of the present invention, a subject or patient includes any mammalian subject or patient, more preferably a human subject or patient.

In a preferred embodiment, therapeutic antibodies that are not intended or required to be depleting to a target cell are antibodies that regulate the function of NK cells by binding to membrane bound molecules on NK Cells. NK cell activity is regulated by a complex mechanism, that involves both stimulating and inhibitory signals. Accordingly, effective NK cell-mediated therapy involving inducing the activation or proliferation of NK cells can be achieved both by a stimulation of these cells or a neutralization of inhibitory signals. Such approaches will be useful in the treatment of tumors or infectious disease, for example, where NK cells mediate target cell lysis, but where the therapeutic antibody binding an NK cell receptor does not contribute to lysis of the NK cell. Other therapies are directed to reducing NK cell activity. These therapies may involve activating inhibitory NK cell receptors or blocking activatory NK cell receptors, and may be useful for the treatment of immune disorders such as autoimmune disorders or in transplantation.

As used herein, “NK” cells refers to a sub-population of lymphocytes that is involved in non-conventional immunity. NK cells can be identified by virtue of certain characteristics and biological properties, such as the expression of specific surface antigens including FcγRIIIa, CD56 and/or CD57, the absence of the alpha/beta or gamma/deka TCR complex on the cell surface, the ability to bind to and kill cells that fail to express “self” MHC/HLA antigens by the activation of specific cytolytic enzymes, the ability to kill tumor cells or other diseased cells that express a ligand for NK activating receptors, and the ability to release protein molecules called cytoldnes that stimulate or inhibit the immune response. Any of these characteristics and activities can be used to identify NK cells, using methods well known in the art.

Within the context of this invention, “active” or “activated” NK cells designate biologically active NK cells, more particularly NK cells having the capacity of lysing target cells. For instance, an “active” NK cell is able to kill cells that express an NK activating receptor-ligand and fails to express “self” MHC/HLA antigens (KIR-incompatible cells). Examples of suitable target cells for use in redirected killing assays are P815 and K562 cells, but any of a number of cell types can be used and are well known in the art (see, e.g., Sivori et al. (1997) J. Exp. Med. 186: 1129-1136; Vitale et al. (1998) J. Exp. Med. 187: 2065-2072; Pessino et al. (1998) J. Exp. Med. 188: 953-960; Neri et al. (2001) Clin. Diag. Lab. Immun. 8:1131-1135). “Active” or “activated” cells can also be identified by any other property or activity known in the art as associated with NK activity, such as cytokine (e.g. IFN-gamma and TNF-alpha production of increases in free intracellular calcium levels.

NK cells are negatively regulated by major histocompatibility complex (MHC) class I-specific inhibitory receptors (Karre et al., 1986; Öhlén et al, 1989; the disclosure of which is incorporated herein by reference). These specific receptors bind to polymorphic determinants of major histocompatibility complex (MHC) class I molecules or HLA and inhibit natural killer (NK) cell lysis. In humans, a family of receptors termed killer Ig-like receptors (KIRs) recognize groups of HLA class I alleles.

In the present invention, the term “an antibody that block(s) the inhibitory receptor of a NK cell” refers to a compound, preferably an antibody or a fragment thereof, specific for an NK cell inhibitory receptor, i.e. KIR or NKG2A/C of NK cells, and neutralizing inhibitory signals of the KIR or NKG2A/C Preferably, the compound, preferably an antibody or a fragment thereof, is able to block the interaction between HLA and an inhibitory receptor of a NK cell. The antibodies may by polyclonal or, preferably, monoclonal. They may be produced by hybridomas or by recombinant cells engineered to express the desired variable and constant domains. The antibodies may be single chain antibodies or other antibody derivatives retaining the antigen specificity and the lower hinge region or a variant thereof such as a Fab fragment, a Fab′2 fragment, a CDR and a ScFv. These may be polyfunctional antibodies, recombinant antibodies, humanized antibodies, or variants thereof.

Preferably, the antibody that blocks the inhibitory receptor of a NK cell is an antibody or a fragment thereof, that neutralizes the inhibitory signal of at least one inhibitory receptor selected in the group consisting of KIR2DL2, KIR2DL3, KIR2DL1, KIR3DL1, KIR3DL2, NKG2A and NKG2C Optionally, the antibody can be selected from the group consisting of GL183 (KIR2DL2, L3, available from Immunotech, France and Beckton Dickinson, USA); EB6 (KIR2DL1, available from Immunotech, France and Beckton Dickinson, USA); AZ138 (KIR3DL1, available from Moretta et al, Univ. Genova, Italy) ; Q66 (KIR3DL2, available from Immunotech, France); Z270 (NKG2A, available from Immunotech, France); P25 (NKG2A/C, available from Moretta et al, Univ. Genova, Italy ; and DX9, Z27 (KIR3DL1, available from Immunotech, France and Beckton Dickinson, USA).

As used herein, the term “activating NK receptor” refers to any molecule on the surface of NK cells that, when stimulated, causes a measurable increase in any property or activity known in the art as associated with NK activity, such as cytokine (e.g. IFN-gamma and TNF-alpha production, increases in intracellular free calcium levels, or the ability to target cells in a redirected killing assay as described, e.g. elsewhere in the present specification. Examples of such receptors include the natural cytotoxicity receptors (NCR) known as NKp3O, NKp44, and NKp46. Another particularly preferred example is the NKG2D receptor, which is also present on CD4+ and CD8+ T cells as well as on gamma-delta T cells. Methods of determining whether an NK cell is active or not are described in more detail below.

Several distinct NK-specific receptors have been identified that play an important role in the NK cell mediated recognition and killing of HLA Class I deficient target cells. These receptors, termed NKp30, NKp46 and NKp44, are members of the Ig superfamily. Their cross-lining, induced by specific mAbs, leads to a strong NK cell activation resulting in increased intracellular C⁺⁺ levels, triggering of cytotoxicity, and lymphokine release. Importantly, mAb-mediated activation of NKp30, NKp46, and/or NKp44 results. in an activation of NK cytotoxicity against many types of target cells. These findings provide evidence for a central role of these receptors in natural cytotoxicity.

Antibodies to NK cell receptors may be produced by any of a variety of techniques known in the art. Typically, they are produced by immunization of a non-human animal, preferably a mouse, with an immunogen comprising an activating receptor present on the surface of NK cells. The activating receptor may comprise entire NK cells or cell membranes, the full length sequence of a receptor such as NKp30 (see, e.g., PCT WO 01/36630, the disclosure of which is herein incorporated by reference in its entirety), NKp44 (see, e.g., Vitale et al. (1998) J. Exp. Med. 187:2065-2072, the disclosure of which is herein incorporated by reference in its entirety), or NKp46 (see, e.g., Sivori et al. (1997) J. Exp. Med. 186:1129-1136; Pessino et al. (1998) J. Exp. Med. 188:953-960; the disclosures of which are herein incorporated by reference in their entireties), or a fragment or derivative thereof, typically an immunogenic fragment, i.e., a portion of the polypeptide comprising an epitope exposed on the surface of the cell expressing any of these receptors, or any other receptor whose stimulation leads to the activation of NK cells. Such fragments typically contain at least 7 consecutive amino acids of the mature polypeptide sequence, even more preferably at least 10 consecutive amino acids thereof. They are essentially derived from the extracellular domain of the receptor. It will be appreciated that any receptor present on the surface of NK cells that, upon stimulation, leads to the activation of the cells as measured by cytotoxicity, increase in intracellular free calcium levels, cytokine production, or any other method known in the art can be used for the generation of antibodies. In preferred embodiments, the activating NK cell receptor used to generate antibodies is a human receptor.

In one embodiment, the immunogen comprises a wild-type human NKp30, NKp44, or NKp46 polypeptide in a lipid membrane, typically at the surface of a cell. In a specific embodiment, the immunogen comprises intact NK cells, particularly intact human NK cells, optionally treated or lysed. Examples of preferred isolated antibodies of the invention include isolated antibodies that are directed against at least one isolated amino acid compound of the invention, and that can induce an increase of at least about 4, preferably at least about 5, more preferably at least about 6 times, of the natural cytotoxicity triggered by a NK cell placed in the presence of a target cell in a 1:1 ratio. In a specific embodiment, the antibody binds essentially the same epitope as any of the following monoclonal antibodies: AZ20, A76, Z25, Z231, or BAB281. Such antibodies are referred to herein as “AZ20-like antibodies,” “A76-like antibodies,” etc. The term “binds to substantially the same epitope or determinant as “the monoclonal antibody x means that an antibody “can compete” with x, where x is AZ20, A76, etc. The identification of one or more antibodies that bind(s) to substantially the same epitope as the monoclonal antibody in question can be readily determined using any one of variety of immunological screening assays in which antibody competition can be assessed. All such assays are routine in the art (see, e.g., U.S. Pat. No. 5,660,827, issued Aug. 26, 1997, which is specifically incorporated herein by reference). It will be understood that actually determining the epitope to which the antibody binds is not in any way required to identify an antibody that binds to the same or substantially the same epitope as the monoclonal antibody in question. In other aspects, an antibody may bind a molecule other than a natural cytotoxicity receptor (NCR) or inhibitor receptor on an NK cell. For example, an antibody may bind any other NK surface molecule, including activating molecules, costimulatory receptors or adhesion molecules, examples including but not limited to NKp80, 2B4 (CD244), NKRP-1A (CD161), BY55 (CD160), PEN5 (CD162R), L-selectin (CD62L) and CD31.

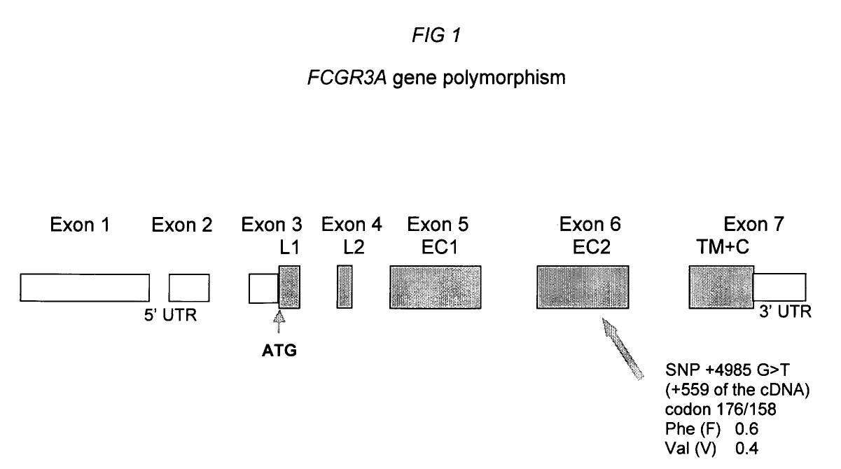

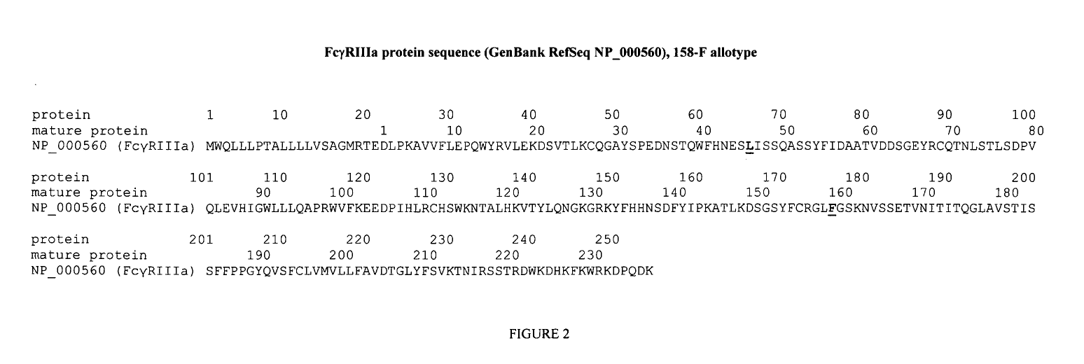

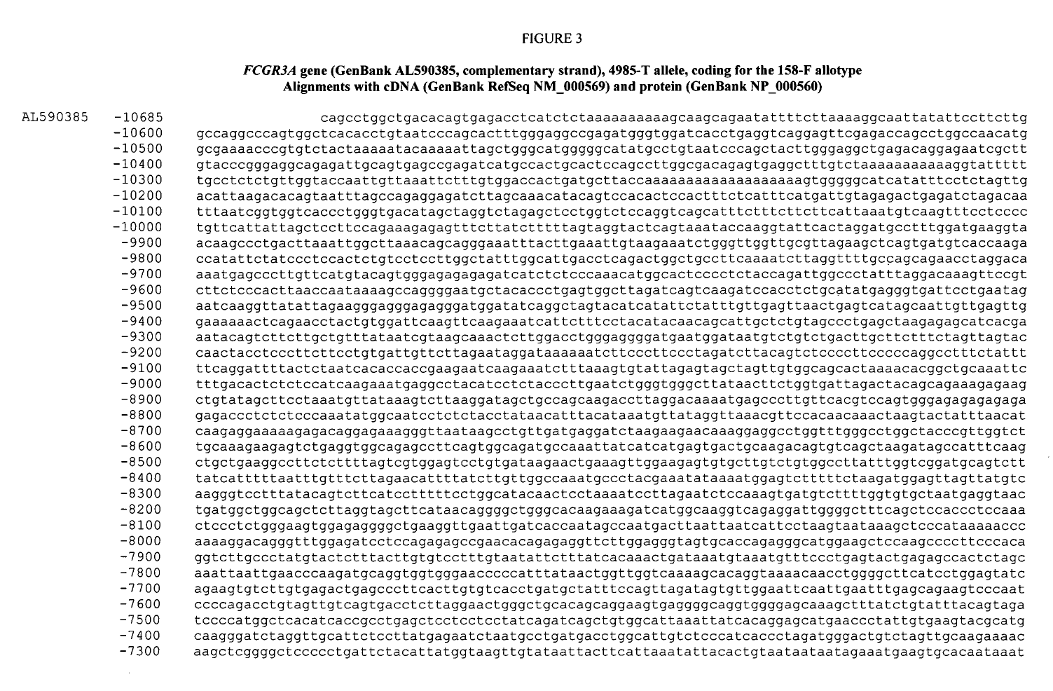

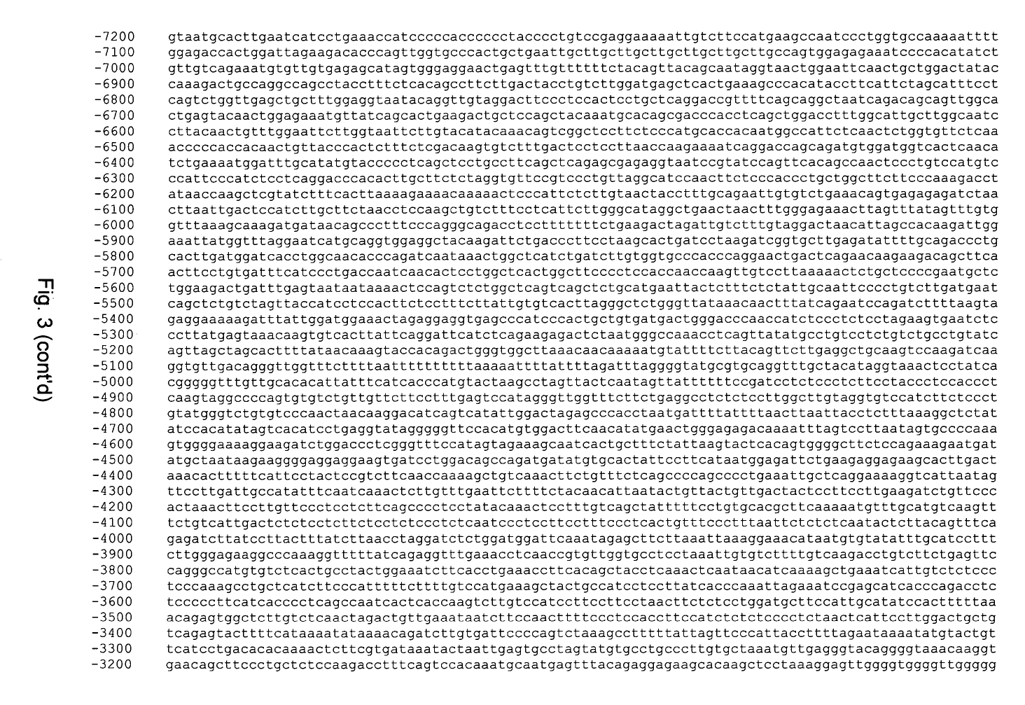

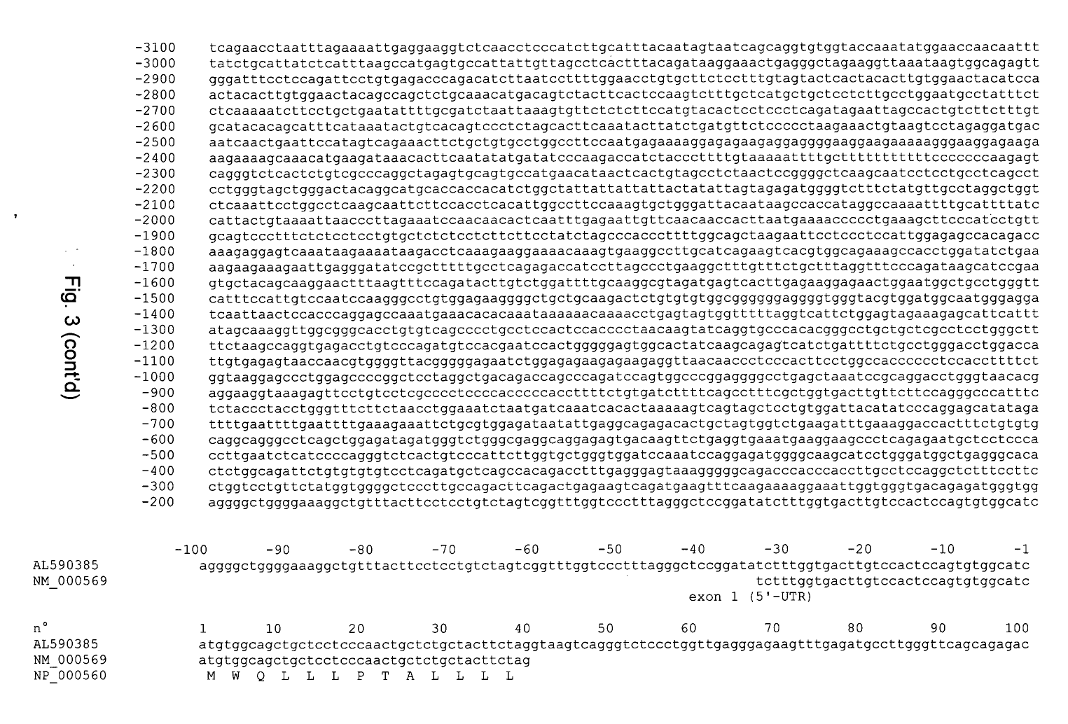

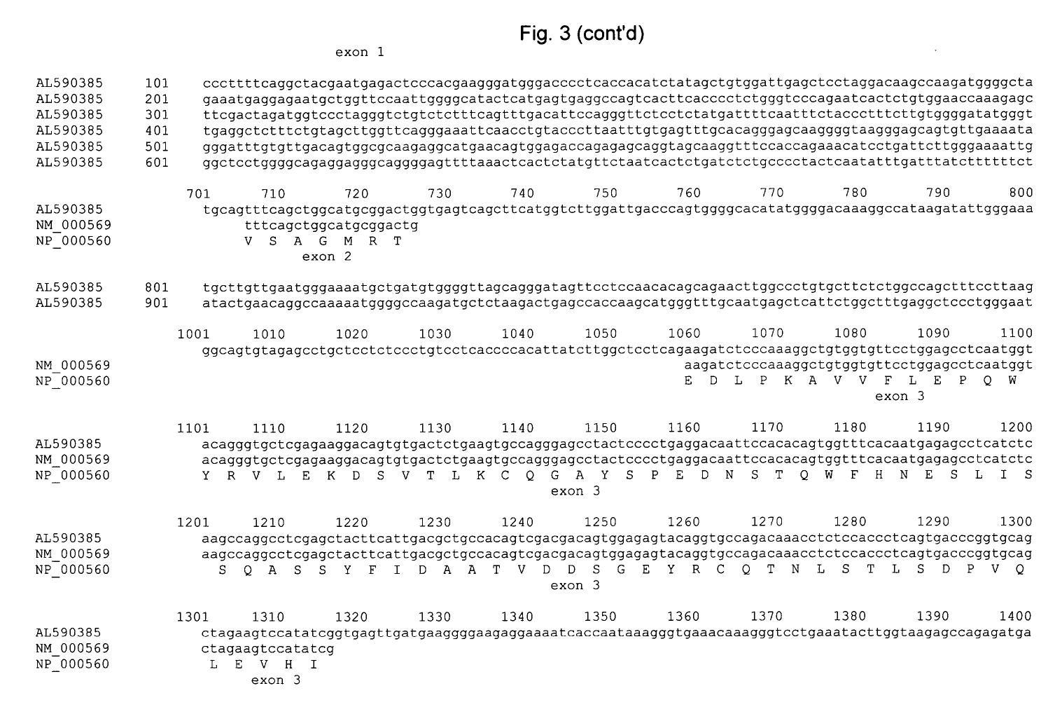

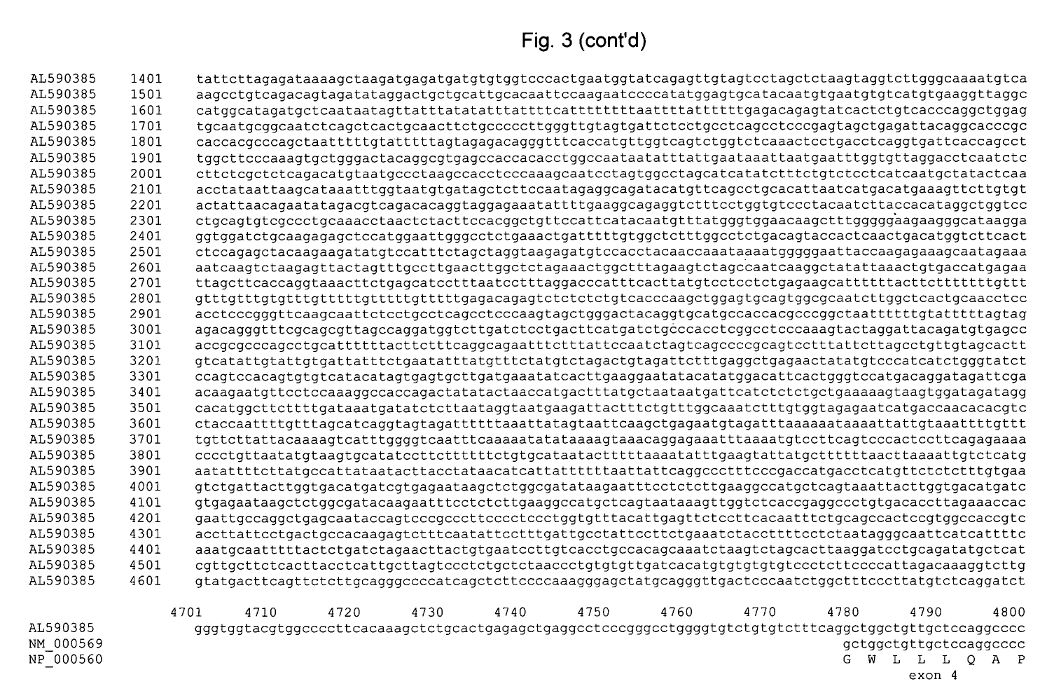

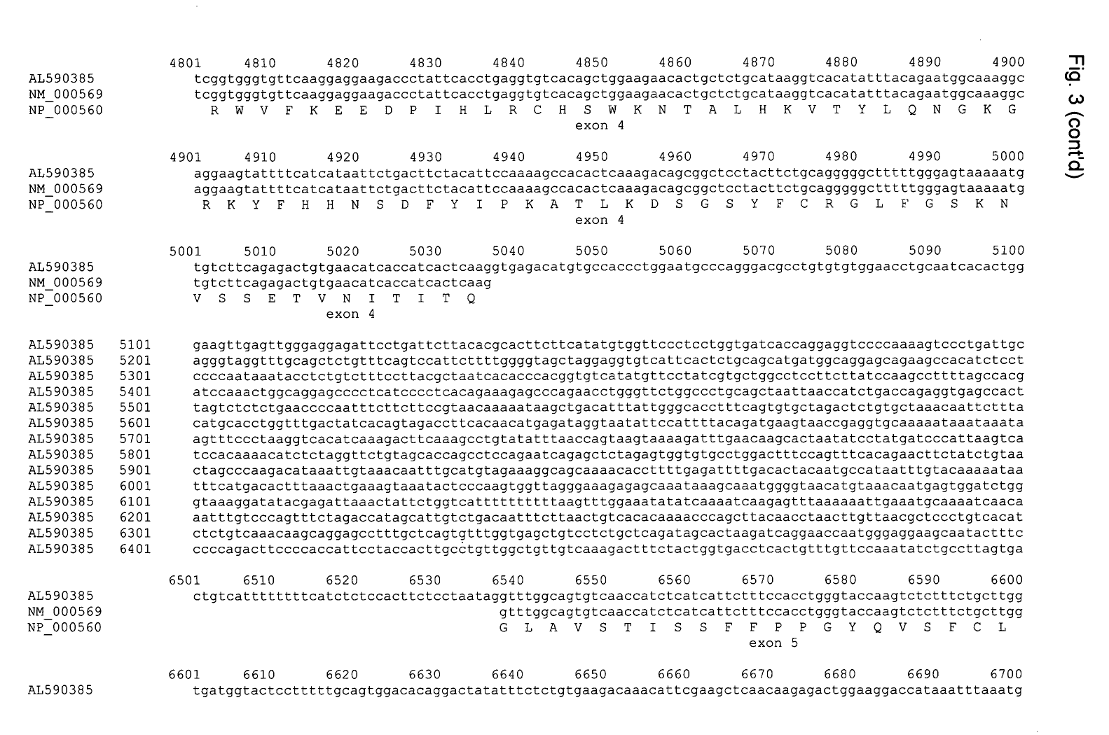

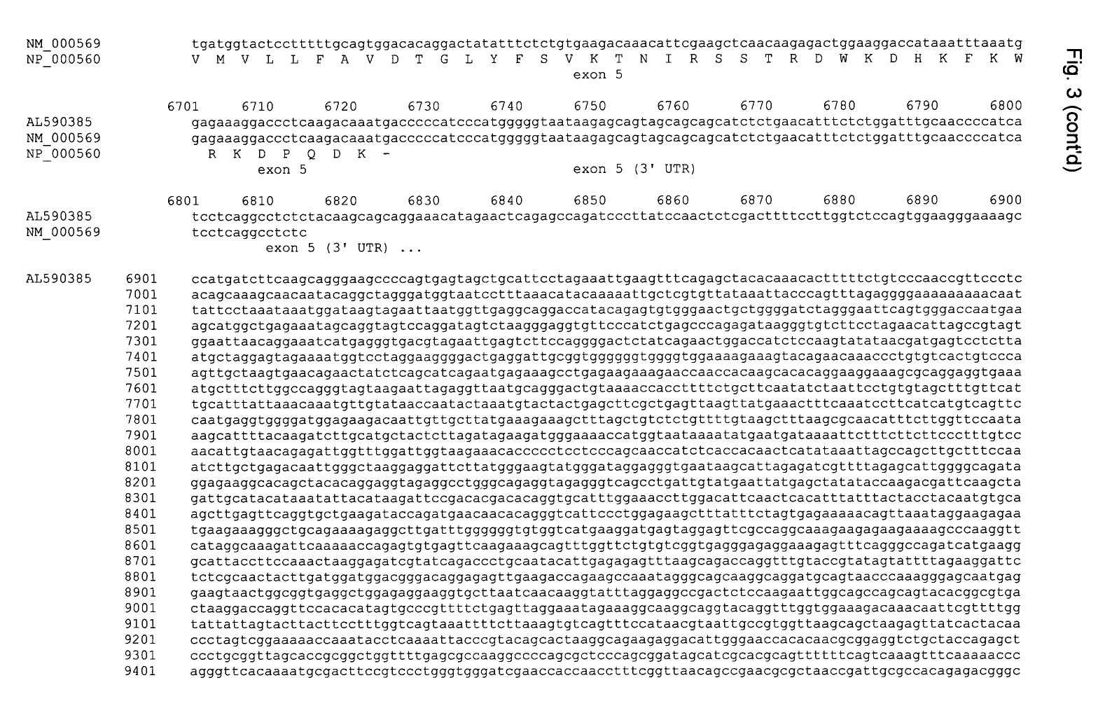

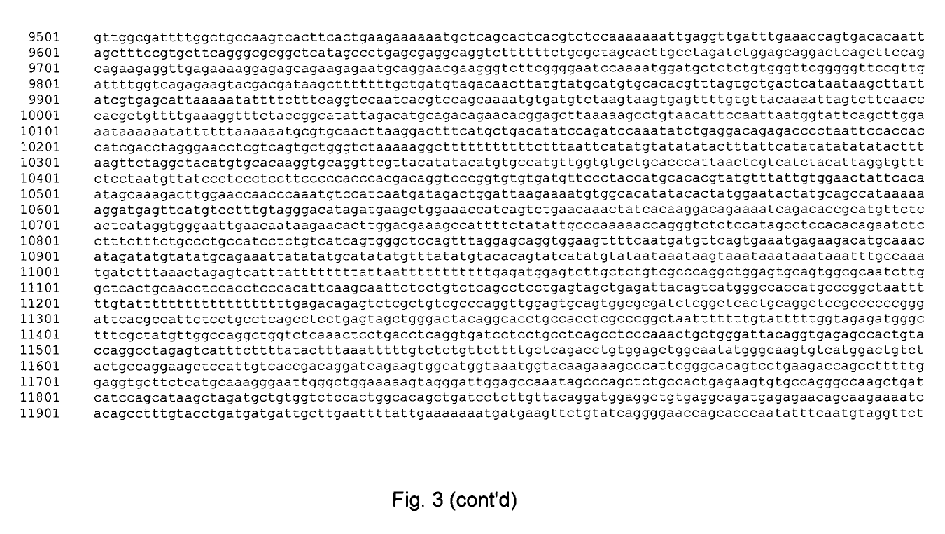

According to the invention the term FCGR3A gene refers to any nucleic acid molecule encoding a FcγRIIIa polypeptide in a subject. This term includes, in particular, genomic DNA, cDNA, RNA (pre-rRNA, messenger RNA, etc.), etc. or any synthetic nucleic acid comprising all or part of the sequence thereof. Synthetic nucleic acid includes cDNA, prepared from RNAs, and containing at least a portion of a sequence of the FCGR3A genomic DNA as for example one or more introns or a portion containing one or more mutations. Most preferably, the term FCGR3A gene refers to genomic DNA, cDNA or mRNA, typically genomic DNA or MRNA. The FCGR3A gene is preferably a human FCGR3A gene or nucleic acid, i.e., comprises the sequence of a nucleic acid encoding all or part of a FcγRIIIa polypeptide having the sequence of human FcγRIIIa polypeptide. Such nucleic acids can be isolated or prepared according to known techniques. For instance, they may be isolated from gene libraries or banks, by hybridization techniques. They can also be genetically or chemically synthesized. The genetic organization of a human FCGR3A gene is depicted on FIG. 1. The amino acid sequence of human FcγRIIIa is represented FIG. 2. Amino acid position 158 is numbered from residue 1 of the mature protein, and corresponds to residue 176 of the pre-protein having a signal peptide. The sequence of a wild type FCGR3A gene is represented on FIG. 3 (see also Genbank accession Number AL590385 or NM_{—}000569 for partial sequence).

Within the context of this invention, a portion or part means at least 3 nucleotides (e.g., a codon), preferably at least 9 nucleotides, even more preferably at least 15 nucleotides, and can contain as much as 1000 nucleotides. Such a portion can be obtained by any technique well known in the art, e.g., enzymatic and/or chemical cleavage, chemical synthesis or a combination thereof. The sequence of a portion of a FCGR3A gene encoding amino acid position 158 is represented below, for sake of clarity.

| | |
|---|---|
| cDNA | 540 550 560 570 580 |
| genomic DNA | 4970 4980 4990 5000. |
| 158F allele | tcctacttctgcagggggctttttgggagtaaaaatgtgtcttca |
| | S Y F C R G L F G S K N V S S |
| 158V allele | tcctacttctgcagggggcttgttgggagtaaaaatgtgtcttca |
| | S Y F C R G L V G S K N V S S |

As indicated throughout the present disclosure, the invention comprises methods comprising determining in vitro the FCGR3A 158 genotype of a subject. It will be appreciated that in any of the embodiments of the invention referring to determining the FCGR3A genotype, it will be readily possible to determine the genotype by determining the phenotype, that is by determining the identity of the amino acid residue present (or encoded) at position 158 of the FcγRIIIa polypeptide. Thus, determining the FCGR3A genotype can comprise or consist of determining the identity of the amino acid residue present (or encoded) at position 158 of the FcγRIIIa polypeptide.

Preferably, homozygosity for a Valine at position 158 of the FcγRIIIa receptor is indicative of a decreased response to said treatment in terms of efficacy, and a phenylalanine at position 158 of the FcγRIIIa receptor (heterozygous or homozygous) is indicative of an increased response to said treatment. With respect to side effects from an antibody treatment, preferably homozygosity for a Valine at position 158 of the FcγRIIIa receptor is indicative of a increased susceptibility to side effects and a phenylalanine at position 158 of the FcγRIIIa receptor is indicative of decreased side effects or susceptibility to side effects to said treatment. The impact of the genotype of the FcγRIIIa receptor at position 158 on the therapeutic response or the side effects is thus generally more strongly marked, that is an decreased response to treatment and/or increased side effects, when the subject is homozygote at position 158 for Valine. However, subjects homozygous or heterozygous for phenylalanine at position 158 have similar responses to treatment, the responses of both of these groups being increased compared to that observed in patients who are homozygous at position 158 for Valine. Subjects homozygous or heterozygous for phenylalanine at position 158 also have similar to side effects from an antibody treatment, the susceptibility to side effects of both of these groups being decreased compared to that observed in patients who are homozygous at position 158 for Valine.

Genotyping the FCGR3A gene or corresponding polypeptide in said subject may be achieved by various techniques, comprising analysing the coding nucleic acid molecules or the encoded polypeptide. Analysis may comprise sequencing, migration, electrophoresis, immuno-techniques, amplifications, specific digestions or hybridisations, etc.

In a particular embodiment, determining amino acid residue at position 158 of FcγRIIIa receptor comprises a step of sequencing the FCGR3A receptor gene or RNA or a portion thereof comprising the nucleotides encoding amino acid residue 158.

In an other particular embodiment, determining amino acid residue at position 158 of FcγRIIIa receptor comprises a step of amplifying the FCGR3A receptor gene or RNA or a portion thereof comprising the nucleotides encoding amino acid residue 158. Amplification may be performed by polymerase chain reaction (PCR), such as simple PCR, RT-PCR or nested PCR, for instance, using conventional methods and primers. A preferred genotyping method, including the disclosure of nucleic acid primers, for determining amino acid residue at position 158 of FcγRIIIa receptor is provided in Dall'Ozzo S, Andres C, Bardos P, Watier H, and Thibault G, J Immunol Methods. 2003 Jun 1;277(1-2):185-92, which disclosure, including but not limited to specific nucleotide sequences disclosed therein, is incorporated herein by reference in its entirety.

In this regard, amplification primers for use in this invention more preferably contain less than about 50 nucleotides even more preferably less than 30 nucleotides, typically less than about 25 or 20 nucleotides. Also, preferred primers usually contain at least 5, preferably at least 8 nucleotides, to ensure specificity. The sequence of the primer can be prepared based on the sequence of the FCGR3A gene, to allow full complementarity therewith, preferably. The probe may be labelled using any known techniques such as radioactivity, fluorescence, enzymatic, chemical, etc. This labeling can use for example Phosphor 32, biotin (16-dUTP), digoxygenin (11-dUTP). It should be understood that the present invention shall not be bound or limited by particular detection or labelling techniques. The primers may further comprise restriction sites to introduce allele-specific restriction sites in the amplified nucleic acids, as disclosed below.

Specific examples of such amplification primers are, for instance, SEQ ID NO. 1-4.

It should be understood that other primers can be designed by the skilled artisan, such as any fragment of the FCGR3A gene, for use in the amplification step and especially a pair of primers comprising a forward sequence and a reverse sequence wherein said primers of said pair hybridize with a region of a FCGR3A gene and allow amplification of at least a portion of the FCGR3A gene containing codon 158. In a preferred embodiment, each pair of primers comprises at least one primer that is complementary, and overlaps with codon 158, and allows to discriminate between 158V (gtt) and 158F (ttt). The amplification conditions may also be adjusted by the skilled person, based on common general knowledge and the guidance contained in the specification.

In a particular embodiment, the method of the present invention thus comprises a PCR amplification of a portion of the FCGR3A mRNA or gDNA with specific oligonucleotide primers, in the cell or in the biological sample, said portion comprising codon 158, and a direct or indirect analysis of PCR products, e.g., by electrophoresis, particularly Denaturing Gel Gradient Electrophoresis (DGGE).

In an other particular embodiment, determining amino acid residue at position 158 of FcγRIIIa receptor comprises a step of allele-specific restriction enzyme digestion. This can be done by using restriction enzymes that cleave the coding sequence of a particular allele (e.g., the 158V allele) and that do not cleave the other allele (e.g., the 158F allele, or vice versa). Where such allele-specific restriction enzyme sites are not present naturally in the sequence, they may be introduced therein artificially, by amplifying the nucleic acid with allele-specific amplification primers containing such a site in their sequence. Upon amplification, determining the presence of an allele maybe carried out by analyzing the digestion products, for instance by electrophoresis. This technique also allows to discriminate subjects that are homozygous or heterozygous for the selected allele.

Examples of allele-specific amplification primers include for instance SEQ ID NO: 3. SEQ ID NO:3 introduces the first 3 nucleotides of the NlaIII site (5′-CATG-3′). Cleavage occurs after G. This primer comprises 11 bases that do not hybridise with FCGR3A, that extend the primer in order to facilitate electrophoretic analysis of the amplification products) and 21 bases that hybridise to FCGR3A, except for nucleotide 31 (A) which creates the restriction site.

In a further particular embodiment, determining amino acid residue at position 158 of FcγRIIIa receptor comprises a step of hybridization of the FCGR3A receptor gene or RNA or a portion thereof comprising the nucleotides encoding amino acid residue 158, with a nucleic acid probe specific for the genotype Valine or Phenylalanine, and determining the presence or absence of hybrids.

It should be understood that the above methods can be used either alone or in various combinations. Furthermore, other techniques known to the skilled person may be used as well to determine the FCGR3A-158 genotype, such as any method employing amplification (e.g. PCR), specific primers, specific probes, migration, etc., typically quantitative RT-PCR, LCR (Ligase Chain Reaction), TMA (Transcription Mediated Amplification), PCE (an enzyme amplified immunoassay and bDNA (branched DNA signal amplification) assays.

In a preferred embodiment of this invention, determining amino acid residue at position 158 of FcγRIIIa receptor comprises:

obtaining genomic DNA from a biological sample,

amplifying the FcγRIIIa receptor gene or a portion thereof comprising the nucleotides encoding amino acid residue 158, and

determining amino acid residue at position 158 of said FcγRIIIa receptor gene.

Amplification can be accomplished with any specific technique such as PCR, including nested PCR, using specific primers as described above. In a most preferred embodiment, determining amino acid residue at position 158 is performed by allele-specific restriction enzyme digestion. In that case, the method comprises:

obtaining genomic DNA from a biological sample,

amplifying the FcγRIIIa receptor gene or a portion thereof comprising the nucleotides encoding amino acid residue 158,

introducing an allele-specific restriction site,

digesting the nucleic acids with the enzyme specific for said restriction site and,

analysing the digestion products, i.e., by electrophoresis, the presence of digestion products being indicative of the presence of the allele.

In an other particular embodiment, the genotype is determined by a method comprising : total (or messenger) RNA extraction from cell or biological sample or biological fluid in vitro or ex vivo, optionally cDNA synthesis, (PCR) amplification with FCGR3A -specific oligonucleotide primers, and analysis of PCR products.

The method of this invention may also comprise determining amino acid residue at position 158 of FcγRIIIa receptor directly by sequencing the FcγRIIIa receptor polypeptide or a portion thereof comprising amino acid residue 158 or by using reagents specific for each allele of the FcγRIIIa polypeptide. This can be determined by any suitable technique known to the skilled artisan, including by immuno-assay (ELISA, EIA, RIA, etc.). This can be made using any affinity reagent specific for a FcγRIIIa158 polypeptide, more preferably any antibody or fragment or derivative thereof. In a particular embodiment, the FcγRIIIa158 polypeptide is detected with an anti-FcγRIIIa158 antibody (or a fragment thereof) that discriminates between FcγRIIIa158V and FcγRIIIa158F, more preferably a monoclonal antibody. The antibody (or affinity reagent) may be labelled by any suitable method (radioactivity, fluorescence, enzymatic, chemical, etc.). Alternatively, FcγRIIIa158 antibody immune complexes may be revealed (and/or quantified) using a second reagent (e.g., antibody), labelled, that binds to the anti-FcγRIIIa158 antibody, for instance.

The above methods are based on the genotyping of FCGR3A158 in a biological sample of the subject. The biological sample may be any sample containing a FCGR3A gene or corresponding polypeptide, particularly blood, bone marrow, lymph node or a fluid, particularly blood or urine, that contains a FCGR3A158 gene or polypeptide. Furthermore, because the FCGR3A 158 gene is generally present within the cells, tissues or fluids mentioned above, the method of this invention usually uses a sample treated to render the gene or polypeptide available for detection or analysis. Treatment may comprise any conventional fixation techniques, cell lysis (mechanical or chemical or physical), or any other conventional method used in immunohistology or biology, for instance.

The method is particularly suited to determine the response of a subject to a therapeutic antibody treatment. In this regard, in a particular embodiment, the subject has an a disorder and the therapeutic antibody treatment aims at ameliorating the disorder, and where the antibody need not be depleting toward a cell to which it is bound. Membrane targets of the antibodies are discussed above.

Further aspects and advantages of this invention will be disclosed in the following examples, which should be regarded as illustrative and not limiting the scope of this application.

### EXAMPLE 1

### Genotyping of FCGR3A-158V/F polymorphism

Materials and Methods

FCGR3A-158V/F Genotyping

All samples are analysed in the same laboratory and DNA is extracted using standard procedures including precautions to avoid cross-contamination. DNA can be isolated from peripheral blood, bone marrow or lymph node. Genotyping of FCGR3A-158V/F polymorphism is performed as described by Koene e al (Koene et al, Blood. 1997;90:1109-1114) with a nested PCR followed by an allele-specific restriction enzyme digestion. Briefly, two FCGR3A specific primers (5′-ATATITACAGAATGGCACAGG-3′, SEQ ID NO: 1; 5′-GACTTGGTACCCAGGTTGAA-3′, SEQ ID NO: 2) (Eurobio, Les Ulis, France) are used to amplify a 1.2 kb fragment containing the polymorphic site. The PCR assay is performed with 1.25 μg of genomic DNA, 200 ng of each primer, 200 μmol/L of each dNTP (MBI Fermentas, Vilnius, Lithuania) and 1 U of Taq DNA polymerase (Promega, Charbonnière, France) as recommended by the manufacturer. This first PCR consists in 10 min at 95° C., then 35 cycles (each consisting in 3 steps at 95° C. for 1 min, 57° C. for 1.5 min, 72° C. for 1.5 min) and 8 min at 72° C. to achieve complete extension. The second PCR used primers (5′-ATCAGATTCFATCCTACTTCTGCAGGGGGCAT-3′ SEQ ID NO: 3; 5′-ACGTGCTGAGCTTGAGTGATGGTGATGTTAC3′ SEQ ID NO: 4) (Eurobio) amplify a 94 bp fragment and create a NlaIII restriction site only in the FCGR3A-158V allele. This nested PCR is performed with 1 μL of the amplified DNA, 150 ng of each primer, 200 μmol/L of each dNTP and 1 U of Taq DNA polymerase. The first cycle consists in 5 min at 95° C. then 35 cycles (each consisting in 3 steps at 95° C. for 1 min, 64° C. for 1 min, 72° C. for 1 min) and 9.5 min at 72° C. to complete extension. The amplified DNA (10 μL) is then digested with 10 U of NlaIII (New England Biolabs, Hitchin, England) for 12 h at 37° C. and separated by electrophoresis on a 8% polyacrylamide gel. After staining with ethidium bromide, DNA bands are visualized with UV light. For homozygous FCGR3A-158F patients, only one undigested band (94 bp) is visible. Three bands (94 bp, 61 bp and 33 bp) are seen in heterozygous individuals whereas for homozygous FCGR3A-158V patients, only two digested bands (61 bp and 33 bp) are obtained.

1-30. (canceled)

31. A method comprising sequencing the FcγRIIIa receptor polypeptide or polynucleotide encoding the FcγRIIIa receptor of a subject and determining amino acid residue at position 158 of said subject's FcγRIIIa receptor;
a) a determination that a subject has a Phenylalanine at position 158 being indicative of said subject's increased responsiveness to a treatment, and a Valine at position 158 being indicative of said subject's decreased responsiveness to said treatment, and wherein said treatment does not require depletion of cells with which the composition is associated or bound;
b) a determination that a subject has a Phenylalanine at position 158 being indicative of said subject's increased responsiveness to a treatment, and a Valine at position 158 being indicative of a decreased response to said treatment, wherein said Fc portion is of the G4 subclass;
c) a determination that a subject has a Phenylalanine at position 158 being indicative of an increased response to said treatment, and a Valine at position 158 being indicative of a decreased response to said treatment, wherein said treatment does not require depletion of cells with which the composition is associated or bound and wherein said determination results in selecting a subject for treatment with a composition comprising an Fc portion, for monitoring a treated subject, or for identifying a subpopulation of treated subjects; or
d) a determination that a subject has a Valine at position 158 of the FcγRIIIa receptor being indicative of the subject's potential for increased side effects or susceptibility to side effects to a treatment and a phenylalanine at position 158 of the FcγRIIIa receptor being indicative of reduced side effects or susceptibility to side effects to a treatment, wherein said treatment does not require depletion of cells with which the composition is associated or bound.

32. The method according to claim 31, wherein said composition comprises an antibody.

33. The method according to claim 31, wherein said composition comprises a fusion protein comprising an Fc portion.

34. The method according to claim 31, wherein said the Fc portion is of the G4 subclass.

35. The method according to claim 31, wherein said treatment is to augment or to reduce an immune response in the subject.

36. The method according to claim 31, wherein said treatment is effective to treat a tumor in the subject.

37. The method according to claim 31, wherein said composition specifically binds an NK cell surface receptor.

38. The method according to claim 31, wherein said composition specifically binds a T cell surface receptor.

39. The method according to claim 38, wherein said NK cell surface receptor is selected from the group consisting of an inhibitory and an activatory cell surface receptor.

40. The method according to claim 39, wherein said NK cell surface receptor is an inhibitory cell surface receptor.

41. The method according to claim 39, wherein said NK cell surface receptor is an activatory cell surface receptor.

42. The method according to claim 41, wherein said activatory cell surface receptor is selected from the group consisting of NKp30, NKp44, NKp46, and NKG2D.

43. The method according to claim 40, wherein said inhibitory cell surface receptor is selected from the group consisting of a KIR and CD94/NKG2A.

44. The method according to claim 31, wherein determining amino acid residue at position 158 of FcγRIIIa receptor comprises a step of sequencing the FcγRIIIa receptor gene or RNA or a portion thereof comprising the nucleotides encoding amino acid residue 158.

45. The method according to claim 31, wherein determining amino acid residue at position 158 of FcγRIIIa receptor comprises a step of amplifying the FcγRIIIa receptor gene or RNA or a portion thereof comprising the nucleotides encoding amino acid residue 158.

46. The method according to claim 45, wherein amplification is performed by polymerase chain reaction (PCR), such as PCR, RT-PCR, and nested PCR.

47. The method according to claim 31, wherein determining amino acid residue at position 158 of FcγRIIIa receptor comprises a step of allele-specific restriction enzyme digestion.

48. The method according to claim 31, wherein determining amino acid residue at position 158 of FcγRIIIa receptor comprises a step of hybridization of the FcγRIIIa receptor gene or RNA or a portion thereof comprising the nucleotides encoding amino acid residue 158, with a nucleic acid probe specific for the genotype Valine or Phenylalanine.

49. The method according to claim 31, wherein determining amino acid residue at position 158 of FcγRIIIa receptor comprises:
Obtaining genomic DNA from a biological sample;
Amplifying the FcγRIIIa receptor gene or a portion thereof comprising the nucleotides encoding amino residue 158; and
determining amino acid residue at position 158 of said FcγRIIIa receptor gene.

50. The method according to claim 31, wherein determining amino acid residue at position 158 of FcγRIIa receptor comprises:
Obtaining genomic DNA from a biological sample;
Amplifying the FcγRIIIa receptor gene or a portion thereof comprising the nucleotides encoding amino acid residue 158;
Introducing an allele-specific restriction site;
Digesting the nucleic acids with the enzyme specific for said restriction site; and
Analyzing the digestion products, i.e., by electrophoresis, the presence of digestion products being indicative of the presence of the allele.

51. The method according to claim 31, wherein determining amino acid residue at position 158 of FcγRIIIa receptor comprises: total (or messenger) RNA extraction from cell or biological sample or biological fluid in vitro or ex vivo, optionally cDNA synthesis, (PCR) amplification with specific FCGRIIIa oligonucleotide primers, and analysis of PCR products.

52. The method according to claim 31, wherein determining amino acid residue at position 158 of FcγRIIIa receptor comprises a step of sequencing the FcγRIIIa receptor polypeptide or a portion thereof comprising amino acid residue 158.

53. The method according to claim 31, wherein the subject is a human subject.

54. The method according to claim 53, wherein the subject has a tumor.

55. The method according to claim 53, wherein the subject has an inflammatory disorder.

56. The method according to claim 26, wherein the subject has a disorder selected from the group consisting of: inflammatory skin diseases; psoriasis; inflammatory bowel diseases; Crohn's disease; ulcerative colitis; adult respiratory distress syndrome; dermatitis; CNS inflammatory disorders; multiple sclerosis; uveitic disorders; allergic conditions; eczema; asthma; conditions involving infiltration of T cells; chronic inflammatory responses; skin hypersensitivity reactions; poison ivy; poison oak; autoimmune diseases; rheumatoid arthritis; systemic lupus erythematosus; diabetes mellitus; multiple sclerosis; Raynaud's syndrome; autoimmune thyroiditis; Sjogren's syndrome; juvenile onset diabetes; immune responses associated with delayed hypersensitivity mediated by cytokines and T-lymphocytes typically found in tuberculosis sarcoidosis, polymyositis, granulomatosis, and vasculitis; pernicious anemia; multiple organ injury syndrome secondary to septicaemia or trauma; autoimmune haemolytic anemia; myethemia gravis; antigen-antibody complex mediated diseases; and all types of transplantation rejection, including graft vs. host or host vs. graft disease.

57. The method according to claim 32, wherein said antibody binds a lymphocyte membrane antigen.
